(19) United States
(12) Patent Application Publication
(10) Pub. No.: US 2015/0209313 A1
(43) Pub. Date: Jul. 30, 2015

(54) USE OF X-RAY CONTRAST MEDIA AND RELATED COMPOSITIONS FOR THE TREATMENT, PREVENTION, REDUCTION OF SEVERITY OF OR DELAY OF THE ONSET OF INFLUENZA

(71) Applicant: 3E THERAPEUTICS COMPANY, La Jolla, CA (US)

(72) Inventor: Elliott C. Lasser, La Jolla, CA (US)

(21) Appl. No.: 14/609,293

(22) Filed: Jan. 29, 2015

Related U.S. Application Data
(60) Provisional application No. 62/038,833, filed on Aug. 18, 2014.
(60) Provisional application No. 61/933,306, filed on Jan. 29, 2014.

(51) Int. Cl. A61K 31/192; A61K 31/167

(52) U.S. Cl. CPC A61K 31/192; A61K 31/167

(57) ABSTRACT

Embodiments disclosed herein relate to certain compositions including X-ray contrast media compounds and/or certain tri-iodated phenyl compounds and methods of using the same for preventing or treating influenza infections.

### RELATED PATENT APPLICATIONS

This patent application claims the benefit of U.S. Provisional Patent Application No. 61/933,306 filed on Jan. 29, 2014, entitled USE OF X-RAY CONTRAST MEDIA AND RELATED COMPOSITIONS FOR THE TREATMENT, PREVENTION, REDUCTION OF SEVERITY OF OR DELAY OF THE ONSET OF INFLUENZA, naming Elliott C. Lasser as inventor, and designated by Attorney Docket No. 021994-0429377, and claims the benefit of U.S. Provisional Patent Application No. 62/038,833 filed on Aug. 18, 2014, entitled USE OF X-RAY CONTRAST MEDIA AND RELATED COMPOSITIONS FOR THE TREATMENT, PREVENTION, REDUCTION OF SEVERITY OF OR DELAY OF THE ONSET OF INFLUENZA, naming Elliott C. Lasser as inventor, and designated by Attorney Docket No. 021994-0431944.

### FIELD

Embodiments of the invention relate to certain compositions including compositions comprising X-ray contrast media compounds and methods of using the same for the treatment, prevention, reduction of severity of or delay of the onset of influenza and or infection with influenza virus. The methods can utilize the compositions described herein as well as other compositions comprising an X-ray contrast media.

### BACKGROUND

X-ray contrast media have been used for many years as radiocontrast reagents in vascular imaging using X-rays. Iodine compounds, specifically triiodinated, completely or incompletely substituted, benzene moieties existing in the form of a monomer or a dimer are among the commonly used X-ray contrast media used in vascular imaging. While the molecules have assumed some structural differences over the years, the basic concept that iodine attached to organic ring structures will impair X-ray penetration remains the same. The X-ray molecules, referred to as “contrast media” have been used to opacify blood vessels, organs, and other parts of the body that have orifices leading externally or are amenable to needle injection.

Influenza virus is a RNA virus belonging to the family Orthomyxoviridae that causes inflammation in the respiratory system. The serotype of the virus is divided into types A, B, and C. Type B and type C are confirmed to infect only human, while type A can infect not only human but also various species of mammals including horse, pig, and others, and also various species of domestic poultry and wild birds. The classification of serotype of type A influenza virus depends on the two kinds of proteins observed on the surface of the virus, which are Hemagglutinin (HA) and Neuraminidase (NA). HA is believed to aid the virus in its attachment on somatic cells, while NA is believed to help the virus invade into cells.

Neuraminidase is believed to cleave the alpha-ketosidic bond between oligosaccharides at cell surfaces and terminal neuraminic acid residues to assist viruses to replicate and proliferate in the infected cells, to exit outside the host cells, and to enter in the respiratory mucosa cells (Mark, V. I. Nature Review 6, 967, 2007; Huberman, K. et al. Virology 214, 294, 1995). Certain influenza therapeutics, oseltamivir and zanamivir have neuraminidase inhibitory activity and are believed to suppress both viral infection and viral release. Recent reports suggest that neuraminidase inhibition may enhance the protective effect of mucous against influenza A virus by suppressing cleavage of sialic acids from glycans in mucous (Cohen, M., et al., Virology Journal 10, 321, 2013).

### SUMMARY

In certain embodiments, the invention provides a method of preventing or slowing infection with influenza virus or treating influenza in a mammal by providing or administering to a mammal in need thereof a composition comprising one or more X-ray contrast media compounds in an amount sufficient to prevent or slow said influenza infection or in an amount sufficient to treat said influenza.

In certain embodiments, the invention provides a method of preventing or slowing infection with influenza virus or treating influenza in a mammal in need thereof by providing or administering to a mammal in need thereof a composition comprising a compound of Formula (I) or Formula (II), or a pharmaceutically acceptable salt or ester thereof, in an amount sufficient to prevent or slow said influenza infection or in an amount sufficient to treat said influenza, wherein Formula (I) has the following structure:

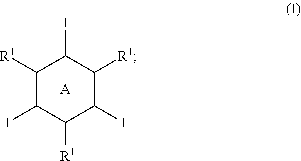

Formula (II) has the following structure:

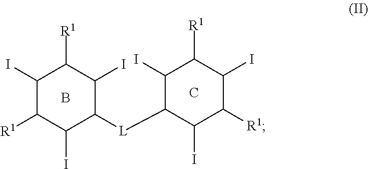

In certain embodiments each R¹ is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl. In certain embodiments L is null or a linker comprising one or more R².

In some embodiments each R² can be independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl, or a combination thereof. The chemical element iodine is often respresented by the symbol I. In some embodiments rings A, B and/or C of Formula (I) and (II) can each independently be aromatic, partially unsaturated or fully saturated. In certain embodiments the compound of Formula (I) and/or (II) is an X-ray contrast media compound.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and therefore, are not to be considered limiting of its scope, the disclosure will be described with additional specificity and detail through use of the accompanying drawings.

FIG. 1 is a chart illustrating the progressive decrease in the level of infection of MDCK cells with Influenza A virus strain A/SD/1/2009(SOIV), in both the presence and absence of human salivary mucins, by X-ray contrast medium Hexabrix in concentrations of 0%, 1%, 10% and 20%.

FIG. 2 is a chart illustrating the degree of inhibition of neuraminidase activity in Influenza A virus strain A/PR/8/34(H1N1) in the presence of buffer, 1 μM oseltamivir, 1% Hexabrix, 10% Hexabrix, and 20% Hexabrix.

FIG. 3 is a chart illustrating the degree of inhibition of neuraminidase activity in Influenza A virus strain A/SD/1/2009(SOIV) in the presence of buffer, 1 μM oseltamivir, 1% Hexabrix, 10% Hexabrix, and 20% Hexabrix.

FIG. 4 is a chart illustrating the degree of inhibition of neuraminidase activity in Influenza A virus strain A/Aichi/2/68(H3N2) in the presence of buffer, 1 μM oseltamivir, 1% Hexabrix, 10% Hexabrix, and 20% Hexabrix.

FIG. 5 graphically illustrates the results of a neuraminidase inhibition assay using an Oseltamivir sensitive strain of influenza (i.e. A/California/07/2009, H1N1). Increasing concentrations of HEXABRIX® (Std1), Visipaque (Std2) and Omnipaque (Std3) were tested (see Table 3) and are shown on the X-axis as percent of undiluted stock (see Table 1). Response values (e.g., average fluorescence) is provided on the Y-axis.

FIG. 6 graphically illustrates the results of a neuraminidase inhibition assay using an Oseltamivir resistant strain of influenza (i.e., A/Maryland/04/2011, H1N1). Increasing concentrations of HEXABRIX® (Std1), Visipaque (Std2) and Omnipaque (Std3) were tested (see Table 4) and are shown on the X-axis as percent of undiluted stock (see Table 1). Response values (e.g., average fluorescence) is provided on the Y-axis.

FIG. 7 graphically illustrates the results of a neuraminidase inhibition assay using an Oseltamivir sensitive strain of influenza (i.e. A/California/07/2009, H1N1). Increasing concentrations of Oseltamivir (Std2) were tested (see Table 5) and are shown on the X-axis. Response values (e.g., average fluorescence) is provided on the Y-axis.

FIG. 8 graphically illustrates the results of a neuraminidase inhibition assay using an Oseltamivir sensitive strain of influenza (i.e. A/Maryland/04/2011, H1N1). Increasing concentrations of Oseltamivir (Std1) were tested (see Table 6) and are shown on the X-axis. Response values (e.g., average fluorescence) is provided on the Y-axis.

FIG. 9 shows the results of a plaque inhibition assay testing the effect of six concentrations of HEXABRIX® (0.9%, 1.88%, 3.25%, 7.5%, 15%, and 30% of stock) shown at the top of each column of wells. The effect of HEXABRIX® on the plaque forming ability of three strains of influenza virus (A/California/07/2009, Top row of 6-well plates; A/Maryland/04/2011; Middle row of 6-well plates; and A/Vietnam/1203/2004, Bottom row of 6-well plates) was visualized by staining infected MDCK cells with MTT for 2 hours.

FIG. 10 shows the results of a plaque inhibition assay testing the effect of six concentrations of Omnipaque (0.9%, 1.88%, 3.25%, 7.5%, 15%, and 30% of stock) shown at the top of each column of wells. The effect of Omnipaque on the plaque forming ability of three strains of influenza virus (A/California/07/2009, Top row of 6-well plates; A/Maryland/04/2011; Middle row of 6-well plates; and A/Vietnam/1203/2004, Bottom row of 6-well plates) was visualized by staining infected MDCK cells with MTT for 2 hours.

FIG. 11 shows the results of a plaque inhibition assay testing the effect of six concentrations of Visipaque (0.9%, 1.88%, 3.25%, 7.5%, 15%, and 30% of stock) shown at the top of each column of wells. The effect of Visipaque on the plaque forming ability of three strains of influenza virus (A/California/07/2009, Top row of 6-well plates; A/Maryland/04/2011; Middle row of 6-well plates; and A/Vietnam/1203/2004, Bottom row of 6-well plates) was visualized by staining infected MDCK cells with MTT for 2 hours.

FIG. 12 shows the results of a plaque inhibition assay testing the effect of HEXABRIX® (Top row of 6-well plates), Omnipaque (Middle row of 6-well plates), and Visipaque (Bottom row of 6-well plates) at six concentrations (0.9%, 1.88%, 3.25%, 7.5%, 15%, and 30% of stock, e.g., shown at the top of each column of wells) on the plaque forming ability of A/California/07/2009 as visualized by MTT staining of infected MDCK cells for 2 hours.

FIG. 13 shows the results of a plaque inhibition assay testing the effect of HEXABRIX® (Top row of 6-well plates), Omnipaque (Middle row of 6-well plates), and Visipaque (Bottom row of 6-well plates) at six concentrations (0.9%, 1.88%, 3.25%, 7.5%, 15%, and 30% of stock, e.g., shown at the top of each column of wells) on the plaque forming ability of A/Maryland/04/2011 as visualized by MTT staining of infected MDCK cells for 2 hours.

FIG. 14 shows the results of a plaque inhibition assay testing the effect of HEXABRIX® (Top row of 6-well plates), Omnipaque (Middle row of 6-well plates), and Visipaque (Bottom row of 6-well plates) at six concentrations (0.9%, 1.88%, 3.25%, 7.5%, 15%, and 30% of stock, e.g., shown at the top of each column of wells) on the plaque forming ability of A/Vietnam/1203/2004 as visualized by MTT staining of infected MDCK cells for 2 hours.

FIG. 15 shows the results of a plaque inhibition assay testing the effect of six concentrations of Oseltamivir (0 μM (i.e. Zero), 1 μM, and 10 μM) shown at the top of each column of wells (panels A, B and C) on the plaque forming ability of three strains of influenza virus (A/California/07/2009, panel A; A/Maryland/04/2011; panel B; and A/Vietnam/1203/2004, panel C) visualized by staining infected MDCK cells with MTT for 2 hours. Panel D shows MTT staining of MDCK cells infected with A/California/07/2009 (top three wells) or A/Maryland/04/2011 (bottom three wells), in the absence of an inhibitor (e.g., in the absence of Oseltamivir). Panel E shows MTT staining of MDCK cells infected with A/Vietnam/1203/2004 in the absence of an inhibitor. Panel F shows a control of MDCK cells grown in the absence of virus.

FIG. 16 shows the results of a plaque inhibition assay testing the effect of HEXABRIX® (Top row of 6-well plates), Omnipaque (Middle row of 6-well plates), and Visipaque (Bottom row of 6-well plates) at six concentrations (0.9%, 1.88%, 3.25%, 7.5%, 15%, and 30% of stock, e.g., shown at the top of each column of wells) on the plaque forming ability of A/Maryland/04/2011 as visualized by MTT staining of infected MDCK cells for 18 hours.

### DETAILED DESCRIPTION

The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

In certain embodiments, the invention provides a method of preventing or slowing infection with an influenza virus or of treating influenza in a mammal, comprising the steps of providing or administering to a mammal in need thereof a composition comprising one or more X-ray contrast media compounds in an amount sufficient to prevent or slow said influenza infection or in an amount sufficient to treat said influenza.

Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model, for example, animal models used for the study of influenza viral infections.

In some embodiments a mammal is “at risk” of an influenza virus infection. A mammal that is at risk may have increased risk factors for an influenza infection, non-limiting examples of which include immunocompromised individuals or immune deficient mammals (e.g., bone marrow transplant recipients, irradiated individuals, mammals having certain types of cancers, particularly those of the bone marrow and blood cells (e.g., leukemia, lymphoma, multiple myeloma), mammals with certain types of chronic infections (e.g., HIV, e.g., AIDS), mammals treated with immunosuppressive agents, mammals suffering from malnutrition and aging, mammals taking certain medications (e.g. disease-modifying anti-rheumatic drugs, immunosuppressive drugs, glucocorticoids) and mammals undergoing chemotherapy), the like or combinations thereof). In some embodiments a mammal at risk is a human over the age of 55, 60 or 65 years. In some embodiments a mammal at risk is a human under the age of 20, 18, 16, 12, 10, 5, 4, 3 or 2 years. In some embodiments a mammal at risk is, will be, or has been in a location or environment suspected of containing an influenza virus. For example, a mammal at risk can be a medical professional that is providing care to another who is suspected of being infected with, or known to be infected with an influenza virus. In certain embodiments, a mammal at risk is any mammal that has been exposed to an influenza virus.

In some embodiments a mammal in need of a treatment or composition described herein is a mammal at risk of an influenza viral infection. In some embodiments a mammal in need of a treatment or composition described herein is infected with, or suspected of being infected with, an influenza virus. In some embodiments a mammal in need of a treatment or composition described herein is a mammal experiencing one or more symptoms associated with an influenza virus infection. Non-limiting examples of symptoms associated with an influenza virus infection include fever (e.g., a body temperature greater than 38.6° C. or 101.5° F.), severe headache, muscle pain, weakness, diarrhea, vomiting, abdominal (stomach) pain, or combinations thereof. In certain embodiments a contrast media or composition described herein is used to treat a symptom of an influenza viral infection.

In certain embodiments, the invention provides a method of preventing or slowing infection with an influenza virus or of treating influenza in a mammal, comprising providing or administering to a mammal in need thereof a composition comprising a compound of Formula (I) or Formula (II) or a pharmaceutically acceptable salt or ester thereof in an amount sufficient to prevent or slow said influenza infection or in an amount sufficient to treat said influenza, wherein Formula (I) has the following structure:

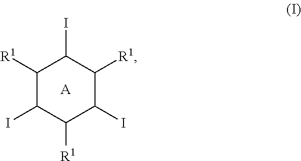

Formula (II) has the following structure:

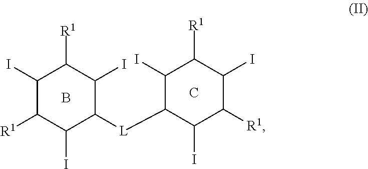

Each R¹ is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl; L is null or a linker comprising one or more R² (for example between 1 and 10 independently selected R² moieties).

In some embodiments each R² is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl, or a combination thereof. The chemical element iodine is often respresented by the symbol I. In some embodiments rings A, B and/or C of Formula (I) and (II) can each independently be aromatic, partially unsaturated or fully saturated.

In certain embodiments, the composition may be administered to a mucous membrane. For example the composition may be administered intranasally or to the mouth, throat, trachea, bronchi and/or lungs. In certain embodiments, the composition is administered according to the invention as an aerosol, mist, spray or nebulized liquid optionally with a device such as an inhaler or nebulizer for providing a measured dose. In certain embodiments, the composition may be administered according to invention topically, orally, buccally, or parenterally. In certain embodiments, the composition may be administered according to the invention as a lozenge.

Each virus subtype has mutated into a variety of strains with differing pathogenic profiles; some are pathogenic to one species but not others, some are pathogenic to multiple species. In certain embodiments, the invention provides methods for preventing or treating influenza where the influenza virus is selected from influenza type A, influenza type B, and influenza type C. Influenza A viruses comprise several strain subtypes (serotypes) which are often labeled according to an immunogenic subtype of H (hemagglutinin, HA) and an immunogenic subtype of N (neuraminidase, NA). There are 18 different known H antigens (H1 to H18) and 11 different known N antigens (N1 to N11). An influenza A virus may comprise any combination of an H subtype and an N subtype and are often named accordingly. In certain such embodiments an influenza virus is type A. In certain embodiments an influenza is of serotype H1N1, H2N2, H3N2, H5N1, H7N7, H1N2, H9N2, H7N2, H7N3, H5N2, or H10N7.

In certain embodiments of the invention, the concentration of the x-ray contrast media compounds in the composition may be measured in terms of mg of iodine per ml. In certain such embodiments the concentration is greater than 50 mg/mL, greater than 10 mg/mL, greater than 150 mg/mL, greater than 200 mg/mL, greater than 250 mg/mL, greater than 300 mg/mL, or greater than 350 mg/mL, or between 50 mg/mL and 150 mg/mL, or between 150 mg/mL and 250 mg/mL, or between 150 mg/mL and 350 mg/mL, or between 250 mg/mL and 350 mg/mL.

In certain embodiments the X-ray contrast compound is of a monomeric form; in other is it is in a dimeric form. In certain embodiments the X-ray contrast compound is of a nonionic type; in others it is of an ionic type.

In certain embodiments the X-ray contrast compound comprise one or more triiodinated, completely or partially substituted, benzene moieties; in certain embodiments two such benzene moieties are linked to form a dimer.

In certain embodiments the X-ray contrast compound is one or more of iopamidol, ioversol, iopromide, iohexol, iothalamate (iothalamic acid), diatrizoate, ioxaglate (ioxaglic acid), iodipamide, iodixanol, iopanoic acid, sodium tyropanoate (BILOPAQUE®), iotrolan, acetrizoate sodium, bunamidiodyl sodium, diatrizoate sodium, iobenzamic acid, iocarmic acid, iocetamic acid, iodamide, iodophthalein sodium, ioglycamic acid, iomeglamic acid, iopental, iophenoxic acid, iopromide, ipronic acid, ioxilan, ipodate, meglumine acetrizoate, meglumine diatrizoate, metrizamide, metrizoic acid, phenobutiodil, phentetiothalein sodium, tyropanoate sodium, and combinations thereof. In certain such embodiments the X-ray contrast compound is one or more of iopamidol, ioversol, iopromide, iohexol, iothalamate (iothalamic acid), diatrizoate, ioxaglate or combinations thereof.

In certain embodiments the invention provides the use of an X-ray media compound or other compound described herein for the preparation of a medicament for the prevention or treatment of influenza.

Embodiments herein relate to methods of preventing, treating or reducing the severity of influenza virus infection or the alleviation of influenza symptoms. Without being bound by any particular theory or limiting the scope of the invention in any way, the following is intended to provide some potential scientific explanations for the useful methods described herein. However, the explanations are not intended to be limiting.

X-ray contrast media compounds have been shown to inhibit the activity of certain enzymes. (Lang, J. and Lasser, E. J, Med Chem 14, 233-236 (1971); Lasser, E. C., et al., Invest. Radiol. 5, 514-517, 1970, “Physiologic Significance of Contrast-Protein Interaction: I. Study In Vitro of Some Enzyme Effects.”) Additionally, X-ray contrast media compounds have been shown, at sufficient concentrations, to inhibit binding of antigens to antibodies and the mechanism for this inhibition is through the binding of X-ray contrast media compounds to the Fc (constant) region of IgE and IgG antibodies. It is believed that such Fc binding is through binding of the X-ray contrast media to carbohydrates or sialic acid moieties attached to such carbohydrates in the Fc region. Consequently, and in view of the recently demonstrated role of viral neuraminidase in binding to cell surface sialic acid moieties in the course of spreading infection, it is expected, as an embodiment of the invention, that X-ray contrast media compounds inhibit the activity of the influenza virus enzyme neuraminidase (NA) and it is hypothesized that use of X-ray contrast media compounds to reduce activity of viral neuraminidase can impair the ability of influenza virus to infect cells. It is hypothesized that X-ray contrast media compounds may impair neuraminidase activity in viral hemmagglutins by interfering with its ability to bind to sialyl moieties on cellular surfaces thereby reducing its ability to infect such cells.

### Formulations

In some embodiments, the composition may be formulated, for example, as a topical formulation. The topical formulation may include, for example, a formulation such as a gel formulation, a cream formulation, a lotion formulation, a paste formulation, an ointment formulation, an oil formulation, and a foam formulation. The composition further may include, for example, an absorption emollient.

In some embodiments, at least part of the affected area of the mammal is contacted with the composition on a daily basis, on an as-needed basis, or on a regular interval such as twice daily, three times daily, every other day, etc. The composition can be administered for a period of time ranging from a single as needed administration to administration for 1 day to multiple years, or any value there between, (e.g., 1-90 days, 1-60 days, 1-30 days, etc.). The dosages described herein can be daily dosages or the dosage of an individual administration, for example, even if multiple administrations occur (e.g., 2 sprays into a nostril).

Some embodiments relate to methods of treating or preventing influenza through administration of invention compositions to the upper respiratory track/bronchi in a mammal in need thereof, for example, by contacting at least part of the upper respiratory tract/bronchi of a mammal with a therapeutically effective amount of a composition as described above or elsewhere herein. The composition can be, for example, formulated as an aerosol formulation, including formulated for use in a nebulizer or an inhaler. The composition further may include other pharmaceutically acceptable components such as a preservative.

In certain embodiments, the methods of the invention can include, for example, providing or administering to a nasal tissue of a mammal a composition that includes an X-ray contrast media compound or a compound according to Formula (I) or Formula (II) or a pharmaceutically acceptable salt or ester thereof in an amount sufficient to prevent or treat an influenza infection or to reduce the severity or onset of an influenza infection.

Other embodiments relate to aerosol compositions that include, for example, a composition as described herein and an aerosolized pharmaceutically acceptable carrier solution or dry powder. The compositions may be formulated, for example, to be substantially absorbed by a bronchus. The compositions also may include, for example, one or more of dichlorodifluoromethane, trichlorofluoromethane, dichlorotetrafluoroethane, carbon dioxide, and the like. The compositions can be formulated for use in a nebulizer or an inhaler, for example.

In certain embodiments, the amount of X-ray contrast media compound can be any sufficient amount to prevent, treat, reduce the severity of, delay the onset of or alleviate a symptom of an influenza infection as contemplated herein. The specific indication as listed herein. In some non-limiting aspects, the X-ray contrast media compound can be provided in a concentration, for example, of 150 Mg I/mL to 350 Mg I/mL. In some aspects the amount or dosage of administered X-ray contrast media compounds can be, for example, from about 0.001 grams to about 200 grams, depending upon the location of delivery and the specific application, for example.

Some embodiments herein are based at least in part upon uses for commercially available X-ray contrast media compound provided by their respective manufacturers.

### X-Ray Contrast Media Compounds

In certain embodiments of the invention, a concentration of an x-ray contrast media compound in a composition is measured in terms of mg of iodine per ml (e.g., mg I/ml). In certain embodiments the concentration is greater than 10 mg/ml, greater than 50 mg/ml, greater than 150 mg/ml, greater than 200 mg/ml, greater than 250 mg/ml, greater than 300 mg/ml, or greater than 350 mg/ml, or between 50 mg/ml and 500 mg/ml, or between 150 mg/ml and 350 mg/ml, or between 150 mg/ml and 250 mg/ml, or between 250 mg/ml and 350 mg/ml.

An X-ray contrast media often comprises an X-ray contrast media compound. In certain embodiments, an X-ray contrast media compound can be ionic or nonionic (or in the case of some dimers, part ionic and part nonionic). In certain embodiments, an X-ray contrast media compound can be monomeric or dimeric, for example. Generally, there can be slight variations in the amide side chains attached at the 3 and 5 positions on the ring and in the nature of the cations (for the ionic media) and there can be slight differences in the length of the aliphatic chains linking the dimers and in the nature of the coupler group. In some aspects an X-ray contrast media compound can be triiodinated. In some aspects an X-ray contrast media compound can be completely or partially substituted benzene moieties. In some aspects an X-ray contrast media compound can be triiodinated, completely or partially substituted, benzene moieties existing in the form of a monomer or a dimer, for example.

In certain embodiments an X-ray contrast compound is of a monomeric form (e.g., comprising a single 6-carbon ring, e.g., Formula (I)). Non-limiting examples of monomeric contrast compounds include diatrizoate, metrizoate, iopamidol, iohexol, ioxilan, iopromide and the like. In certain embodiments an X-ray contrast compound is of a dimeric form (e.g., as depicted in Formula (II)). Non-limiting examples of dimeric contrast compounds include ioxaglate, iodixanol and the like. In certain embodiments the X-ray contrast compound is of a nonionic type. Non-limiting examples of nonionic contrast compounds include iopamidol, iohexol, ioxilan, iopromide, iodixanol and the like. In certain embodiments an X-ray contrast compound is of an ionic type. Non-limiting examples of ionic contrast compounds include diatrizoate, metrizoate, ioxaglate, and the like. Some non-limiting examples of X-ray contrast media compounds that are nonionic monomers include iopamidol, ioversol, iopromide, and iohexol. Some non-limiting examples of X-ray contrast media compounds that are ionic monomers include iothalamate and diatrizoate. Some non-limiting examples of X-ray contrast media compounds that are ionic dimers include ioxaglate (e.g., HEXABRIX®) and iodipamide, while iodixanol (e.g., VISIPAQUE™) and iotrolan are examples of nonionic dimers. In certain embodiments an X-ray contrast media compound is a monomeric compound of general Formula (I) that comprises an ionic contrast media compound. In certain embodiments an X-ray contrast media is a monomeric compound of general Formula (I) that comprises a non-ionic contrast media compound. In certain embodiments an X-ray contrast media compound is a dimeric compound of general Formula (II) that comprises an ionic contrast media compound. In certain embodiments an X-ray contrast media compound is a dimeric compound of general Formula (II) that comprises a non-ionic contrast media compound.

In certain embodiments an X-ray contrast media does not include a monomeric compound of general Formula (I) that comprises an ionic contrast media compound. In some embodiments an X-ray contrast media does not include a monomeric compound of general Formula (I) that comprises a non-ionic contrast media compound. In some embodiments an X-ray contrast media does not include a dimeric compound of general Formula (II) that comprises an ionic contrast media compound. In some embodiments an X-ray contrast media does not include a dimeric compound of general Formula (II) that comprises a non-ionic contrast media compound.

In certain embodiments an X-ray contrast compound comprises one or more triiodinated, completely or partially substituted, benzene moieties existing in the form of a monomer or a dimer, for example. In certain embodiments two such benzene moieties are linked to form a dimer. Generally, there can be slight variations in the amide side chains attached at the 3 and 5 positions on the ring and in the nature of the cations (for the ionic media) and there can be slight differences in the length of the aliphatic chains linking the dimers and in the nature of the coupler group.

Any suitable X-ray contrast media or X-ray contrast media compound can be used in the methods and compositions described herein. In certain embodiments an X-ray contrast compound is one or more of iopamidol, ioversol, iopromide, iohexol, iothalamate (iothalamic acid), diatrizoate, ioxaglate (ioxaglic acid, e.g., HEXABRIX®), iodipamide, iodixanol (e.g., VISIPAQUE™), iopanoic acid, sodium tyropanoate (BILOPAQUE®), iotrolan, acetrizoate sodium, bunamidiodyl sodium, diatrizoate sodium, iobenzamic acid, iocarmic acid, iocetamic acid, iodamide, iodophthalein sodium, ioglycamic acid, iomeglamic acid, iopental, iophenoxic acid, ipronic acid, ioxilan, ipodate, meglumine acetrizoate, meglumine diatrizoate, metrizamide, metrizoic acid, phenobutiodil, phentetiothalein sodium, tyropanoate sodium, the like, or combinations thereof. In certain such embodiments the X-ray contrast compound is one or more of iopamidol, ioversol, iopromide, iohexol, iothalamate (iothalamic acid), diatrizoate, ioxaglate or combinations thereof. The formulas and structures of the above-listed X-ray contrast media compounds can be found in a variety of sources, including for example The Merck Index (Twelfth Edition 1996), which is incorporated herein by reference in its entirety.

### Compositions

Some embodiments relate to compositions comprising one or more X-ray contrast media compounds, including one or more of the X-ray contrast media compounds listed herein. In some aspects, a commercial X-ray contrast media composition or formulation comprising any of the compounds listed above can be used with the possible addition of pharmaceutically acceptable ingredients chosen to adjust the concentration and facilitate the chosen mode of delivery. For example, a commercial X-ray contrast media compound, without being limited thereto, can be one or more of HEXABRIX®, VISIPAQUE (iodixanol), OMNIPAQUE (iohexol), CONRAY®, ISOVIST™, OPTIRAY®, CHOLOGRAFIN®, ISOVUE®, ANGIOVIST™.

The compositions further can include other excipients, carriers and materials as detailed more fully below.

Some embodiments relate to methods of treating a patient suffering from or at risk of suffering from an influenza virus infection. Some embodiments relate to combination therapies utilizing compositions and methods as described herein to be used (e.g., administered together or separately) in combination with other anti-virals or treatments for influenza.

In some embodiments, the compositions for use according to the methods of the invention can include a carrier. The carrier can affect the solubility and/or the diffusivity of the composition compared to the solubility and/or the diffusivity of the compositions for use according to the methods of the invention. Some other embodiments relate to aerosolized compositions for use according to the methods of the invention. The aerosolized compositions can provide a mechanism of obtaining a high surface area of contact between the composition and the upper airway system. In other embodiments, the formulation can be an inhaled powder cut in such a fashion that it will be predisposed to coat the bronchi rather than areas higher or lower in the respiratory system and will not be itself an irritant.

Accordingly, some embodiments relate to methods and compositions to prevent or treat, reduce the severity of, delay the onset of or alleviate a symptom of an influenza infection by administration to the lungs, bronchial passages, trachea, esophagus, sinuses, nasal passages. Methods of administering a pharmaceutical composition to the lungs, bronchial passages, trachea and/or esophagus are known, non-limiting examples of which include intranasal administration, intratracheal instillation, oral inhalative administration (e.g., by use of an inhaler, e.g., single/-multiple dose dry powder inhalers, nebulizers, and the like).

In some embodiments, the compositions for use according to the methods of the invention can include oral compositions. Such compositions can include, for example, a carrier that improves the absorption of the composition through the esophagus, for example, as compared to the absorption of the X-ray contrast media compound alone. The oral composition can also include a thickener that prolongs the transit of the X-ray contrast media compound through the esophagus, for example.

In some embodiments, the compositions for use according to the methods of the invention can include pharmaceutical carriers, fillers, diluents, etc. as described herein, particularly those that are suitable for administration topically, intranasally, orally (including buccal), by inhalation, etc. In some aspects, the X-ray contrast media compound can be formulated with a topical carrier. The topical carrier can increase the absorption via the region of administration (e.g., percutaneous, nasal, buccal, etc.) of the X-ray contrast media compound composition. In some embodiments when administered, for example, via the nose, the compositions can be administered into one or both nostrils in any suitable amount or concentration as described herein.

For nasal administration, the compositions for use according to the methods of the invention can be administered via a nasal spray, drip, gel, etc. or any other suitable form.

The compositions for use according to the methods of the invention can be formulated as described herein. The administered dosage can be any suitable amount, including any described herein. For example, the composition can be provided or administered in a dosage in an amount of 50 μl to 1000 ml, 50 μl to 500 ml, 50 μl to 200 ml, 100 μl to 5 ml, 100 μl to 3 ml, 800 μl to 1,600 μl, 100 μl to 1000 μl, or 100 μl to 500 μl, or any other suitable amount that is effective to prevent, treat, reduce the severity of delay the onset of or alleviate a symptom of an influenza infection.

In some aspects, any one of, or certain of, the contrast media described herein can be specifically excluded from the methods and compositions.

### Route of Administration and Formulation

The exact formulation and route of administration for the compositions for use according to the methods of the invention described herein can be chosen by the individual physician or medical care giver in view of the patient's condition. See e.g., Fingl et al. 1975, in “The Pharmacological Basis of Therapeutics,” Ch 1 p. 1; which is incorporated herein by reference in its entirety. As set forth more fully below, non-limiting examples of routes of administration include, for example, topical, oral, rectal, parenteral delivery (including intramuscular, subcutaneous, injections), as well as, intranasal, or ocular injections. As mentioned above, U.S. Provisional Application Ser. No. 60/914,642, filed on Apr. 27, 2007, and U.S. Provisional Application Ser. No. 60/981,093, filed on Nov. 28, 2007 by Elliott C. Lasser and both entitled “COMPOSITIONS AND METHODS FOR THE TREATMENT OF INFLAMMATORY CONDITIONS,” are each incorporated herein by reference in their entirety. In particular, the Appendix to the specification (from Remington's Pharmaceutical Sciences) is incorporated herein for all of the various formulations, ingredients, excipients, etc., listed therein. The various X-ray contrast materials listed herein, alone or in combination, can be incorporated into or used with the materials described in Remington's.

A composition comprising an X-ray contrast media compound as described herein can be administered to a mammal by any suitable caregiver or medical care professional (e.g., a doctor, physician, nurse, physician's assistance, technician or the like). In some embodiments a composition comprising an X-ray contrast media compound is provided to a mammal (e.g., a human) for self-administration, or is provided to a caregiver for administering to a mammal having, suspected of having, or at risk of having an influenza virus infection.

In certain embodiments one can administer compositions for use according to the methods of the invention in a local rather than systemic manner, for example, via direct application to the skin, mucous membrane or region of interest for treating, including using a depot or sustained release formulation.

In some embodiments, a contrast media can be administered alone. For example, in some embodiments a method of treating a mammal having an influenza virus infection, suspected of having an influenza virus infection or at risk of havening an influenza virus infection comprises providing or administering to the mammal a composition consisting of an X-ray contrast media compound in an amount sufficient to prevent or slow said influenza virus infection or in an amount sufficient to treat said influenza. In other embodiments, a contrast media can be administered in combination with one or more additional materials, for example, as two separate compositions or as a single composition where the additional material(s) is (are) mixed or formulated together with the contrast media. For example, without being limited thereto, a contrast media can be formulated with additional excipients, additional active ingredients, and/or with other contrast media. In some aspects, when administered in the forms described herein a contrast media can attain concentrations at a target tissue such as the nose, mucous membranes, the bronchi, the skin, etc. that cannot be attained by the usual intravascular administration of a contrast material.

The pharmaceutical compositions can be manufactured by any suitable manner, including, e.g., by means of conventional mixing, dissolving, granulating, dragee-making, levigating, emulsifying, encapsulating, entrapping or tableting processes.

Pharmaceutical compositions for use in accordance with the invention thus can be formulated in any suitable manner using one or more physiologically acceptable carriers comprising excipients, additives and/or auxiliaries, which facilitate processing of the active compounds into preparations which can be used pharmaceutically. Proper formulation can depend upon the route of administration chosen. Any suitable techniques, carriers, and excipients can be used, including those understood in the art; e.g., in Remington's Pharmaceutical Sciences, above. The pages in the attached Appendix from Remington's Pharmaceutical Sciences are incorporated herein by reference in their entirety, including without limitation for all of the types of formulations, methods of making, etc.

### Formulations

Compositions for use according to the methods of the invention can be, in some embodiments, aerosolized compositions. The aerosolized composition can be formulated such that the composition has increased solubility and/or diffusivity. The composition can comprise a carrier. The carrier can improve the absorption of the composition, change the viscosity of the composition, change the solubility of the composition, or change the diffusivity of the composition as compared to that of the X-ray contrast media compound alone.

Liquid pharmaceutically administrable compositions can, for example, be prepared by dissolving, dispersing, etc. an X-ray contrast media compound as defined above and optional pharmaceutical adjuvants in a carrier (e.g., water, saline, aqueous dextrose, glycerol, glycols, ethanol or the like) to form a solution or suspension. Solutions to be aerosolized can be prepared in any suitable form, for example, either as liquid solutions or suspensions, as emulsions, or in solid forms suitable for dissolution or suspension in liquid prior to aerosol production and inhalation.

For administration by inhalation, the compositions described herein can conveniently be delivered in the form of an aerosol (e.g., through liquid nebulization, dry powder dispersion or meter-dose administration The aerosol can be delivered from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

By non-limiting example water-based liquid formulations can include an X-ray contrast media compound alone or with non-encapsulating water soluble excipients. Simple formulations can also include organic-based liquid formulations for nebulization or meter-dose inhaler. By non-limiting example organic-based liquid formulations can include an X-ray contrast media compound or with non-encapsulating organic soluble excipients.

Simple formulations can also include dry powder formulations for administration with a dry powder inhaler. By way of non-limiting example, dry powder formulations can include a X-ray contrast media compound alone or with either water soluble or organic soluble non-encapsulating excipients with or without a blending agent such as lactose.

Formulations can include water-based liquid formulations for nebulization. Non-limiting examples of water-based liquid complex formulations can include X-ray contrast media compound encapsulated or complexed with water-soluble excipients such as lipids, liposomes, cyclodextrins, microencapsulations, and emulsions.

Formulations can also include organic-based liquid formulations for nebulization or meter-dose inhaler. Non-limiting examples of organic-based liquid complex formulations can include X-ray contrast media compound encapsulated or complexed with organic-soluble excipients such as lipids, microencapsulations, and reverse-phase water-based emulsions.

Formulations can also include low-solubility, water-based liquid formulations for nebulization. A non-limiting example low-solubility, water-based liquid complex formulations can include X-ray contrast media compound as a low-water soluble, stable nano suspension alone or in co-crystal/co-precipitate excipient complexes, or mixtures with low solubility lipids, such as lipid nano suspensions.

Formulations can also include low-solubility, organic-based liquid formulations for nebulization or meter-dose inhaler. A non-limiting example low-solubility, organic-based liquid complex formulations can include X-ray contrast media compound as a low-organic soluble, stable nano suspension alone or in co-crystal/co-precipitate excipient complexes, or mixtures with low solubility lipids, such as lipid nano suspensions.

Formulations can also include dry powder formulations for administration using a dry powder inhaler. A non-limiting example, complex dry powder formulations can include an X-ray contrast media compound in co-crystal/co-precipitate/spray dried complex or mixture with low-water soluble excipients/salts in dry powder form with or without a blending agent such as lactose.

Specific methods for simple and complex formulation preparation are described herein. Any suitable X-ray contrast media compound, composition or formulation described herein can be directly administered as an aerosol to the respiratory tract.

Any suitable device technology can be used to deliver, for example, a dry powder or a liquid aerosolized product comprising an X-ray contrast material. Dry powder formulations in some circumstances can require less time for drug administration. Liquid formulations can have longer administration times.

For aqueous and other non-pressurized liquid systems, a variety of nebulizers (including small volume nebulizers) can be used to aerosolize the formulations. Compressor-driven nebulizers can utilize jet technology and can use compressed air to generate the liquid aerosol. Such devices are commercially available from, for example, Healthdyne Technologies, Inc.; Invacare, Inc.; Mountain Medical Equipment, Inc.; Pari Respiratory, Inc.; Mada Medical, Inc.; Puritan-Bennet; Schuco, Inc., DeVilbiss Health Care, Inc.; and Hospitak, Inc. Ultrasonic nebulizers generally rely on mechanical energy in the form of vibration of a piezoelectric crystal to generate respirable liquid droplets and are commercially available from, for example, Omron Healthcare, Inc. and DeVilbiss Health Care, Inc. Vibrating mesh nebulizers rely upon either piezoelectric or mechanical pulses to generate respirable liquid droplets. Commercial examples of nebulizers that RESPIRGARD II®, AERONEB®, AERONEB® PRO, and AERONEB® GO produced by Aerogen; AERX® and AERX ESSENCE™ produced by Aradigm; PORTA-NEB®, FREEWAY FREEDOM™, Sidestream, Ventstream and I-neb produced by Respironics, Inc.; and PARI LC-PLUS®, PARI LC-STAR®, and e-Flow7m produced by PARI, GmbH. By further non-limiting example, U.S. Pat. No. 6,196,219, is hereby incorporated by reference in its entirety.

In some embodiments, the drug solution can be formed prior to use of the nebulizer by a patient. In other embodiments, the drug can be stored in the nebulizer in solid form. In this case, the solution can be mixed upon activation of the nebulizer, such as described in U.S. Pat. No. 6,427,682 and PCT Publication No. WO 03/035030, both of which are hereby incorporated by reference in their entirety. In these nebulizers, the drug, optionally combined with excipients to form a solid composition, can be stored in a separate compartment from a liquid solvent.

### Pharmaceutical Carriers

Carriers are often included in a composition to facilitate the incorporation or absorption of a compound into a body, organ, cells or tissues. In some embodiments carriers are included in a composition to prolong the biological half-life of a compound. In certain embodiments, carriers can be conjugated directly to a compound or carriers can be non-covalently associated with a compound. In some embodiments dimethyl sulfoxide (DMSO) is a commonly utilized carrier as it facilitates the uptake of many organic compounds into the cells or tissues of an organism. In some embodiments, a pharmaceutical carrier for a composition described herein can be selected from castor oil, ethylene glycol, monobutyl ether, diethylene glycol monoethyl ether, corn oil, dimethyl sulfoxide, ethylene glycol, isopropanol, soybean oil, glycerin, zinc oxide, titanium dioxide, glycerin, butylene glycol, cetyl alcohol, and sodium hyaluronate.

In certain embodiments comprising hydrophobic excipients, additives, X-ray contrast media compounds or other components, a pharmaceutical carrier for certain of such hydrophobic compounds can be a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant POLYSORBATE 80™, and 65% w/v polyethylene glycol 300, made up to volume in absolute ethanol. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of POLYSORBATE 80™; the fraction size of polyethylene glycol can be varied; other biocompatible polymers can replace polyethylene glycol, e.g., polyvinyl pyrrolidone; and other sugars or polysaccharides can substitute for dextrose.

Alternatively, other delivery systems for hydrophobic pharmaceutical compounds, excipients, or additives can be employed, if required. Liposomes and emulsions are well known examples of delivery vehicles or carriers for hydrophobic drugs and drug compositions. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. The pharmaceutical compositions described herein can be administered to a patient per se, or in pharmaceutical compositions where they are mixed with other active ingredients, as in combination therapy, or suitable carriers or excipient(s). The compounds and compositions can be formulated with salts or excipients, such as for example, sodium or meglumine. Techniques for formulation and administration of the compounds of the instant application can be found in “Remington's Pharmaceutical Sciences,” Mack Publishing Co., Easton, Pa., 18th edition, 1990.

Furthermore, the compounds and compositions used herein can be stable over an extended period of time, for example on the order of months or years. Compositions comprising an X-ray contrast media compound can, in some embodiments, comprise a preservative. The preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®).). The preservative can comprise an alkyl-mercury salt of thiosalicylic acid, such as thiomersal, phenylmercuric nitrate, phenylmercuric acetate or phenylmercuric borate. The preservative can comprise a parabens, such as methylparaben or propylparaben. The preservative can comprise an alcohol, such as chlorobutanol, benzyl alcohol or phenyl ethyl alcohol. The preservative can comprise a biguanide derivative, such as chlorohexidine or polyhexamethylene biguanide. The preservative can comprise sodium perborate, imidazolidinyl urea, and/or sorbic acid. The preservative can comprise stabilized oxychloro complexes, such as known and commercially available under the trade name PURITE®). The preservative can comprise polyglycol-polyamine condensation resins, such as known and commercially available under the trade name POLYQUART®). from Henkel KGaA. The preservative can comprise stabilized hydrogen peroxide generated from a source of hydrogen peroxide for providing an effective trace amount of resultant hydrogen peroxide, such as sodium perborate tetrahydrate. The preservative can be benzalkonium chloride.

The preservative can enable a composition comprising an X-ray contrast media compound to be used on multiple occasions. The preservative can reduce the effects of one or more of acid exposure, base exposure, air exposure, heat, and light on an X-ray contrast media compound. The compounds and compositions used herein can include any suitable buffers, such as for example, sodium citrate buffer and/or sequestering agents, such as edetate disodium sequestering agent. Ingredients, such as meglumine, may be added to adjust the pH of a composition or compound described herein. Compounds and compositions described herein may comprise sodium and/or iodine, such as organically bound iodine. Compositions and compounds used herein may be provided in a container in which the air is replaced by another substance, such as nitrogen.

### Dosages and Products

Certain embodiments provide pharmaceutical compositions suitable for use in the technology, which include compositions where the active ingredients are contained in an amount effective to achieve its intended purpose. A “therapeutically effective amount” means an amount to prevent, treat, reduce the severity of, delay the onset of or inhibit a symptom of an influenza infection. The symptom can be a symptom already occurring or expected to occur. Determination of a therapeutically effective amount is well within the capability of those skilled in the art, especially in light of the detailed disclosure provided herein.

In other embodiments, a therapeutically effective amount can describe the amount necessary for a significant quantity of the composition to contact the desired region or tissue where prevention or treatment of an influenza infection is desired.

Within certain embodiments of the invention, dosages of administered X-ray contrast media compounds can be from 0.001-200 grams, or 0.001-50 grams, 0.001-10 grams, etc. In some aspects the amount can be, for example, from 0.001-0.1 grams, 0.1-5 grams, 5-10 grams, 10-15 grams, 15-20 grams, 20-25 grams, 25-30 grams, 30-35 grams, 35-40 grains, 40-45 grams, 45-50 grams and 50-200 grams. In some non-limiting aspects, an X-ray contrast media compound can be provided in a concentration, for example, of 1 mg I/ml to 1000 mg I/ml, 50 mg I/ml to 500 mg I/ml, or 150 mg I/ml to 350 mg I/ml. In certain embodiments the contrast media described herein can be administered at a concentration of about 100 mg I/ml, about 150 mg I/ml, about 200 mg I/ml, about 150 mg I/ml, about 200 mg I/ml, about 250 mg I/ml, about 300 mg I/ml, about 350 mg I/ml, about 400 mg I/ml, about 450 mg I/ml, or about 500 mg I/ml.

Volumes suitable for intravenous administration are well known. For example, 50 ml-100 ml of contrast media (e.g., ioxaglate, ioversol or iodixanol) at a working concentration of between about 200 mg I/ml to about 350 mg I/ml can be safely administered intravenously to an adult human subject. In some embodiments the amount delivered can be any suitable amount, for example, in order to contact the desired tissue in a therapeutically effective manner. As one non-limiting example, the compositions can be delivered to the nose, and the amount delivered to each nostril can be from about 50 microliters to about 500 microliters, or about 100, 150, 200, 250, 300, 350, 400, 450 or about 500 microliters, for example. In certain embodiments the compositions can be delivered to the nose, and the amount delivered to each nostril can be from about 20 microliters to about 1500 microliters, 50 microliters to about 1000 microliters, or 50 microliters to about 500 microliters. In some embodiments, composition described herein can be delivered to the nose in a volume of about 50, 100, 200, 300, 400 or 500 microliters for example.

The compositions can, if desired, be presented in a pack or dispenser device, which can contain one or more unit dosage forms containing the active ingredient. The pack can for example comprise metal or plastic foil, such as a blister pack. The pack or dispenser device can be accompanied by instructions for administration. The pack or dispenser can also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert. Compositions comprising a compound of the invention formulated in a compatible pharmaceutical carrier can also be prepared, placed in an appropriate container, and labeled for treatment of an indicated condition.

In some embodiments the compositions, formulations, combination products and materials described herein can be included as part of kits, which kits can include one or more of the compositions, X-ray contrast media compounds, formulations of the same, combination drugs and products and other materials described herein. In some embodiments the products, compositions, kits, formulations, etc. can come in an amount, package, product format with enough medication to treat a patient for 1 day to 1 year, 1 day to 180 days, 1 day to 120 days, 1 day to 90 days, 1 day to 60 days, 1 day to 30 days, or any day or number of days there between.

### Kits

In some embodiments the compositions, formulations, combination products and materials described herein can be included as part of kits, which kits can include one or more of the compositions, X-ray contrast media compounds, formulations of the same, combination drugs and products and other materials described herein. In some embodiments the products, compositions, kits, formulations, etc. can come in an amount, package, product format with enough medication to treat a patient for 1 day to 1 year, 1 day to 180 days, 1 day to 120 days, 1 day to 90 days, 1 day to 60 days, 1 day to 30 days, or any day or number of days there between.

The invention provides kits including contrast media of the invention, combination compositions and pharmaceutical formulations thereof, packaged into suitable packaging material. A kit optionally includes a label or packaging insert including a description of the components or instructions for use in vitro, in vivo, or ex vivo, of the components therein. Exemplary instructions include instructions for a method, treatment protocol or therapeutic regimen.

A kit can contain a collection of such components, e.g., two or more conjugates alone, or in combination with another therapeutically useful composition (e.g., an anti-proliferative or immune-enhancing drug). The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics (PK) and pharmacodynamics (PD). Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Kits can additionally include other components. Each component of the kit can be enclosed within an individual container and all of the various containers can be within a single package. Invention kits can be designed for cold storage. Invention kits can further be designed to contain host cells expressing fusion polypeptides of the invention, or that contain nucleic acids encoding fusion polypeptides. The cells in the kit can be maintained under appropriate storage conditions until the cells are ready to be used. For example, a kit including one or more cells can contain appropriate cell storage medium so that the cells can be thawed and grown.

### EXAMPLES

### Example 1

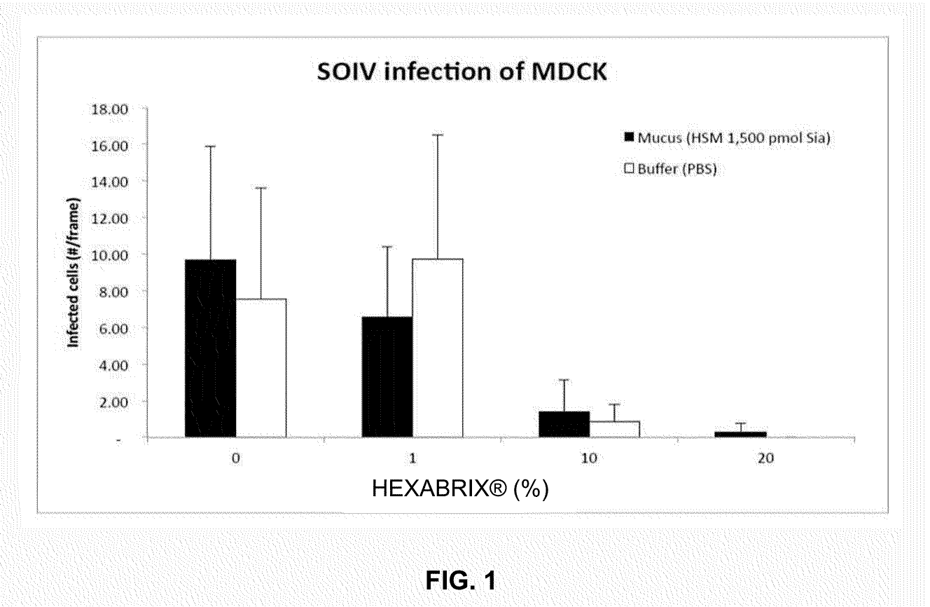

MDCK cells were challenged with a swine origin influenza virus, specifically Influenza A virus strain A/SD/1/2009(SOIV), with and without the presence of an X-ray contrast media, Hexabrix® (Ioxaglic acid). A monolayer of MDCK cells was then layered with either buffer (PBS) or purified human salivary mucins (HSM) containing 0%, 1%, 10% or 20% Hexabrix. A dose dependent reduction in the number of infected cells was observed with an EC50 at less than 10% Hexabrix as shown in FIG. 1.

### Example 2

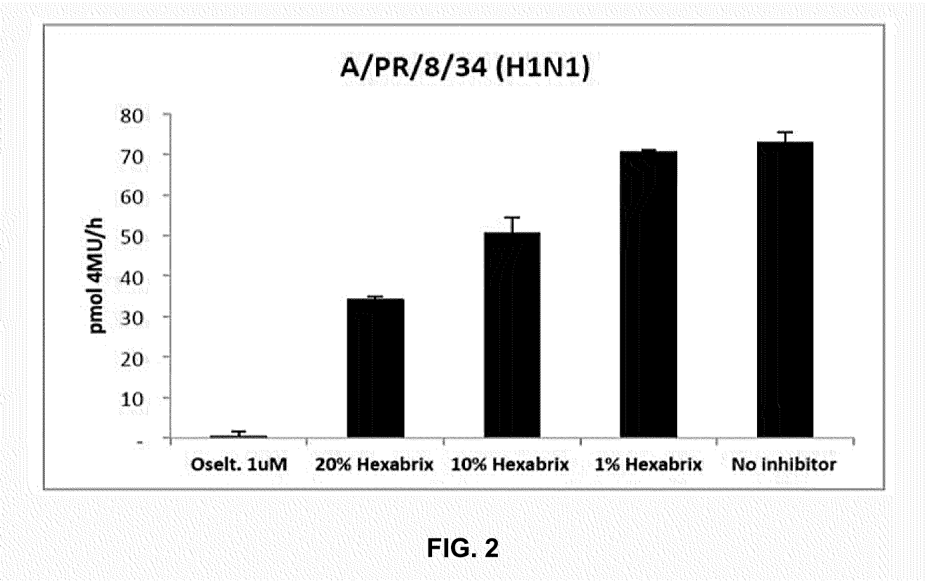

Inhibition of viral neuraminidase activity in Influenza A strain A/PR/8/34(H1N1) (ATCC) was measured using 2′-(4-methylumbelliferyl)-α-D-N-acetylneuraminic acid (4MU-Neu5AC) (Sigma-Aldrich) where cleavage of the Neu5AC sialic acid from the 4MU compound yields fluorescent signal which permits the quantitative measurement of the amount of picomoles of 4MU in an hour. Neuraminidase activity of this H1N1 strain was measured in the presence of buffer, 1 μM oseltamivir, 1% Hexabrix, 10% Hexabrix, and 20% Hexabrix. Dose-dependent reduction in neuraminidase activity was observed as shown in FIG. 2.

### Example 3

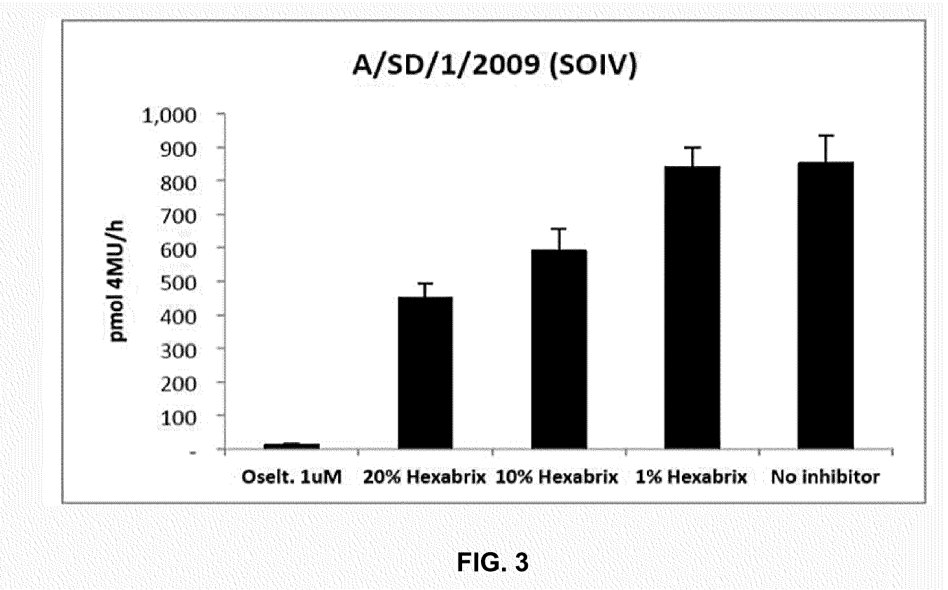

Inhibition of viral neuraminidase activity in Influenza A strain A/SD/1/2009(SOIV) was measured using 2′-(4-methylumbelliferyl)-α-D-N-acetylneuraminic acid (4MU-Neu5AC) (Sigma-Aldrich) where cleavage of the Neu5AC sialic acid from the 4MU compound yields fluorescent signal which permits the quantitative measurement of the amount of picomoles of 4MU in an hour. Neuraminidase activity of this SOIV strain was measured in the presence of buffer, 1 μM oseltamivir, 1% Hexabrix, 10% Hexabrix, and 20% Hexabrix. Dose-dependent reduction in neuraminidase activity was observed as shown in FIG. 3.

### Example 4

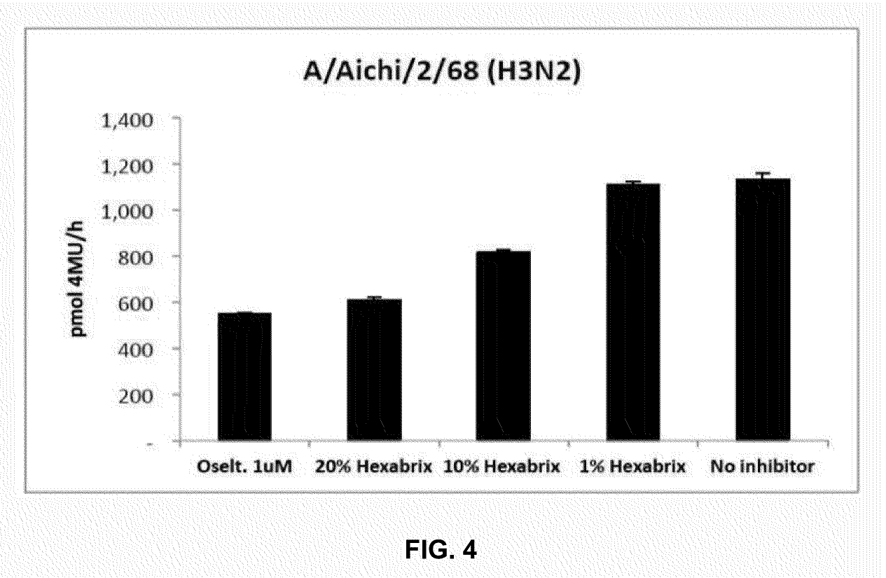

Inhibition of viral neuraminidase activity in Influenza A strain A/Aichi/2/68(H3N2) (ATCC) was measured using 2′-(4-methylumbelliferyl)-α-D-N-acetylneuraminic acid (4MU-Neu5AC) (Sigma-Aldrich) where cleavage of the Neu5AC sialic acid from the 4MU compound yields fluorescent signal which permits the quantitative measurement of the amount of picomoles of 4MU in an hour. Neuraminidase activity of this SOIV strain was measured in the presence of buffer, 1 μM oseltamivir, 1% Hexabrix, 10% Hexabrix, and 20% Hexabrix. Dose-dependent reduction in neuraminidase activity was observed as shown in FIG. 4.

### Example 5

Ioxaglic acid (HEXABRIX®), Iodixanol (Vispaque) and Iohexol (Omnipaque) were tested in a Neuraminidase Inhibition Assay (NIA) to determine an IC50 (half maximal inhibitory concentration concentration) of each X-ray contrast media (CM). The Neuraminidase Inhibition Assay was performed as described in Wetherall et al. (N. T. Wetherall et al, J. of Clinical Micro. (2003) 41:742-750) and as described herein. The assay was performed in a black 96-well plate using fluorogenic substrate 2′-(4-methylumbelliferyl)-α-d-N-acetylneuraminic acid (MUNANA). Seven two-fold dilutions of each CM were made in duplicate and mixed with a fixed volume of virus with known infectivity titer. After incubation, the substrate MUNANA was added and the enzymatic reaction allowed to proceed for one hour. The reaction was stopped with high pH buffer and fluorescence was immediately read by a fluorometer. Raw data was analyzed for IC50 using the MASTERPLEX® ReaderFit program by Hitachi. Oseltamivir (Oseltamivir Phosphate, TAMIFLU®) was included in the assay and the IC50 of Oseltamivir was determined against both an Oseltamivir sensitive and Oseltamivir resistant H1N1 virus.

The CM samples were stored in clear glass or plastic bottles at room temperature until use. The three stock CM samples tested are shown in Table 1 below.

TABLE 1
Stock CM Samples

| | | Starting |
|---|---|---|
| Sample | Details | Concentration |
| HEXABRIX ® | Tyco Healthcare, Mallinckrodt, | 100% |
| (Ioxaglic acid) | Cat. 5505-21, Lot A0231, | |
| | Exp January 2014, 200 ml | |
| Visipaque | GE Healthcare, Cat. NDC 0407222316, | 100% |
| (iodixanol) | Lot 12288359, Exp 08 Nov. 2016, | |
| | 50 ml | |
| Omnipaque | GE Healthcare, Cat. 0407141310, Lot | 100% |
| (iohexol) | 12212550, Exp 27 Sep. 2016, 10 ml | |

Dilutions of each CM sample were made with enzyme reaction buffer, substrate and virus at concentrations of 37.5, 18.8, 9.4, 4.7, 2.3, 1.2, and 0.6% of the stock contrast medias shown in Table 1.

Two strains of influenza virus were tested in the presence or absence of each CM media and in the presence or absence of Oseltamivir. The two influenza strains are identified below:
Influenza Virus A/California/07/2009 H1N1, Sensitive to Oseltamivir, CDC ID 2009712112, VIRAPUR Lot G1121C1
Influenza Virus A/Maryland/04/2011 H1N1, Resistant to Oseltamivir, H275Y, CDC ID 2011767959, VIRAPUR Lot E1218B

H1N1 influenza viruses A/Maryland/04/2011 is known to be oseltamivir resistant, and A/California/07/2009 is known to be oseltamivir sensitive. The H1N1 virus strains used in this study were grown in chick allantoic cavities and titered for the appropriate dilution to use in the NIA assay. The dilution of virus that was used gave maximal fluorescent counts of approximately 65,000-80,000 with a high signal-to-noise ratio.

### Results

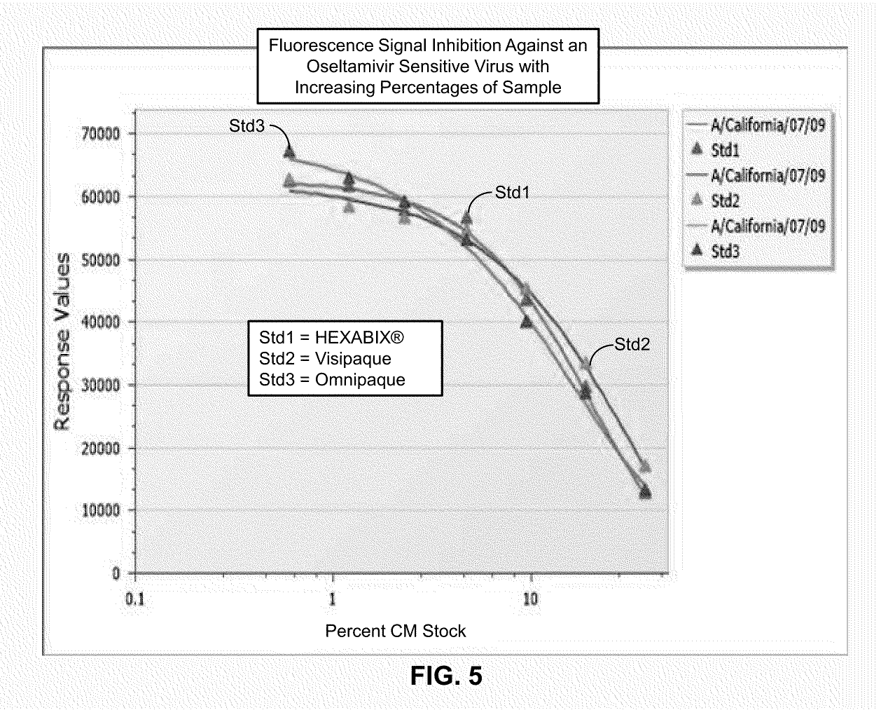

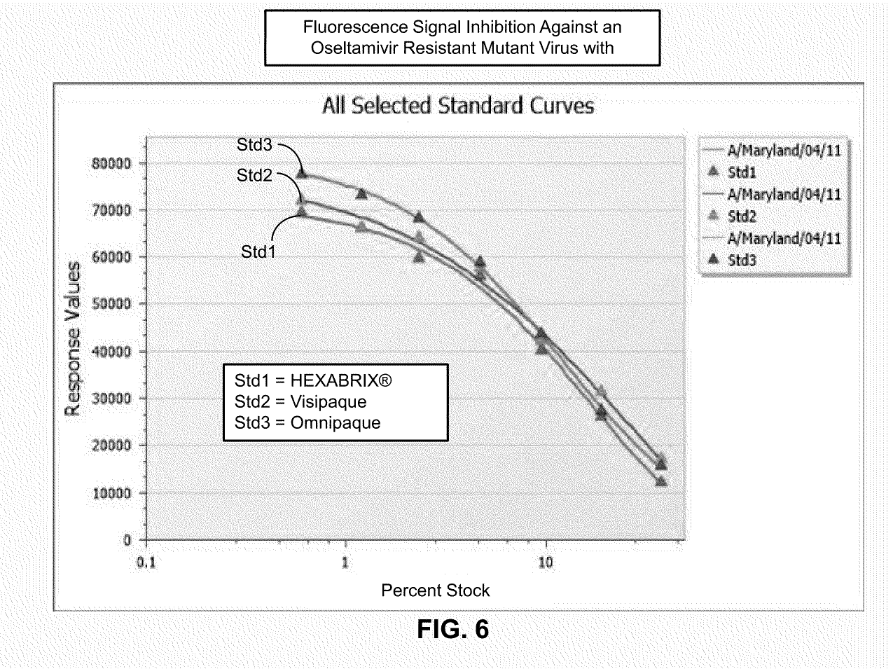

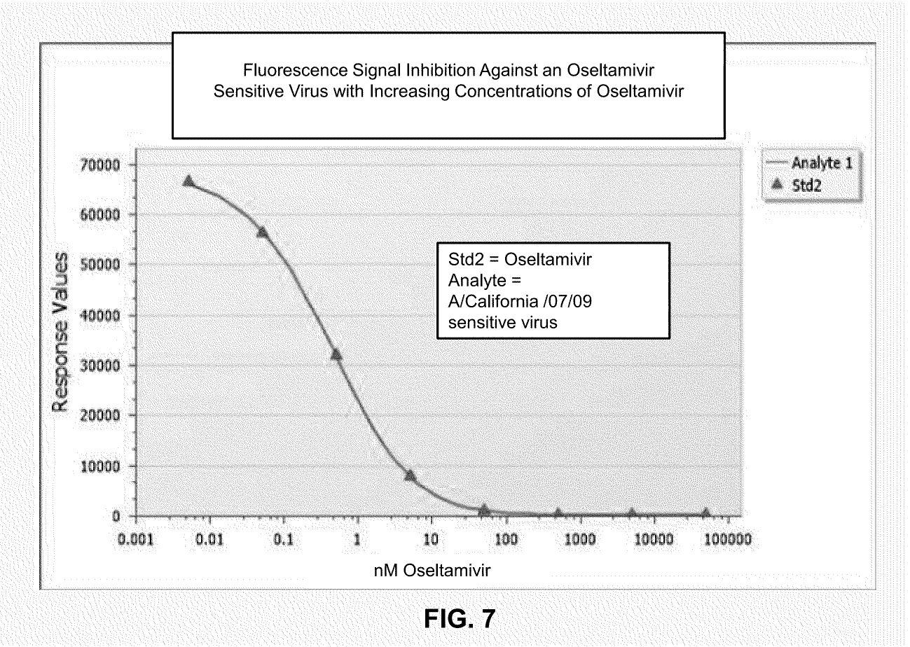

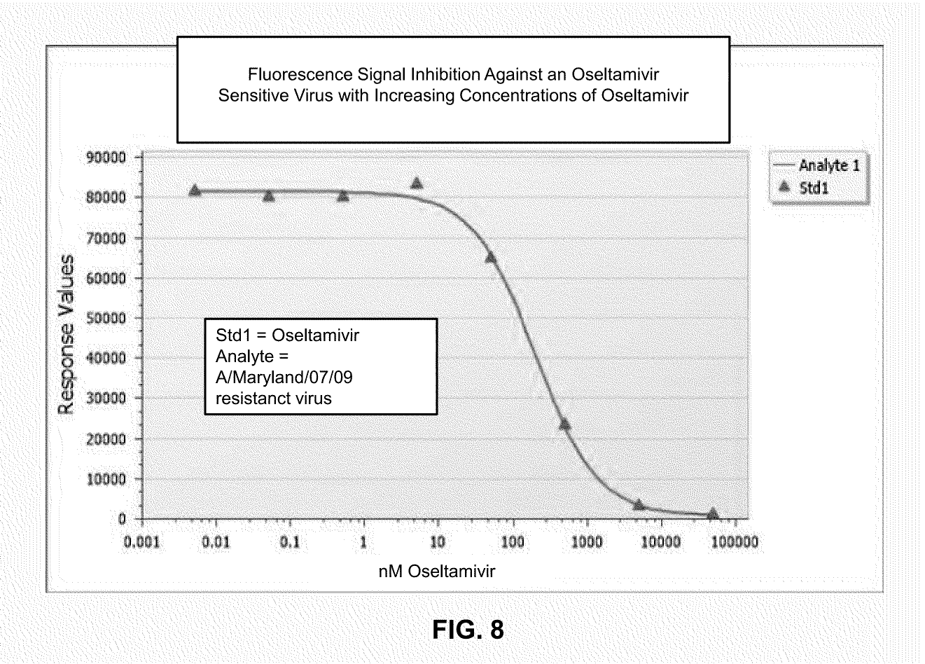

Average fluorescent counts and calculated IC50 values for each CM sample when tested with A/California/07/2009 are shown in Table 2 and the inhibition response curves are graphically illustrated in FIG. 5. Average fluorescent counts and calculated IC50 values for each CM sample when tested with A/Maryland/04/2011 are shown in Table 3 and the inhibition response curves are graphically illustrated in FIG. 6. Average fluorescent counts and calculated IC50 values for Oseltamivir when tested with A/California/07/2009 are shown in Table 4 and the inhibition response curve is graphically illustrated in FIG. 7. Average fluorescent counts and calculated IC50 values for Oseltamivir when tested with A/Maryland/04/2011 are shown in Table 5 and the inhibition response curve is graphically illustrated in FIG. 8.

TABLE 2
Neuraminidase Inhibition of HEXABRIX ®, Visipaque and Omnipaque
on A/California/07/2009

| | Sample Name/ | Average | Percent Drug | EC50/IC50 |
|---|---|---|---|---|
| Standard | Concentration | Fluorescence | (%) | (% Drug) |
| A1 | HEXABRIX ®/ | 12508.00 | 37.50 | 17.04 |
| | 37.5% | | | |
| B1 | HEXABRIX ®/ | 29400.00 | 18.80 | |
| | 18.8% | | | |
| C1 | HEXABRIX ®/ | 43266.00 | 9.40 | |
| | 9.4% | | | |
| D1 | HEXABRIX ®/ | 56384.00 | 4.70 | |
| | 4.7% | | | |
| E1 | HEXABRIX ®/ | 57720.00 | 2.30 | |
| | 2.3% | | | |
| F1 | HEXABRIX ®/ | 61475.00 | 1.20 | |
| | 1.2% | | | |
| G1 | HEXABRIX ®/ | 62367.00 | 0.60 | |
| | 0.6% | | | |
| H1 | HEXABRIX ®/ | 67439.00 | 0.00 | |
| | 0.0% | | | |
| A3 | Visipaque/ | 16886.00 | 37.50 | 20.59 |
| | 37.5% | | | |
| B3 | Visipaque/ | 33163.00 | 18.80 | |
| | 18.8% | | | |
| C3 | Visipaque/ | 45049.00 | 9.40 | |
| | 9.4% | | | |
| D3 | Visipaque/ | 54368.00 | 4.70 | |
| | 4.7% | | | |
| E3 | Visipaque/ | 56518.00 | 2.30 | |
| | 2.3% | | | |
| F3 | Visipaque/ | 58108.00 | 1.20 | |
| | 1.2% | | | |
| G3 | Visipaque/ | 62489.00 | 0.60 | |
| | 0.6% | | | |
| H3 | Visipaque/ | 66434.00 | 0.00 | |
| | 0.0% | | | |
| A5 | Omnipaque/ | 13044.00 | 37.50 | 13.17 |
| | 37.5% | | | |
| B5 | Omnipaque/ | 28518.00 | 18.80 | |
| | 18.8% | | | |
| C5 | Omnipaque/ | 39814.00 | 9.40 | |
| | 9.4% | | | |
| D5 | Omnipaque/ | 52932.00 | 4.70 | |
| | 4.7% | | | |
| E5 | Omnipaque/ | 58910.00 | 2.30 | |
| | 2.3% | | | |
| F5 | Omnipaque/ | 62770.00 | 1.20 | |
| | 1.2% | | | |
| G5 | Omnipaque/ | 67129.00 | 0.60 | |
| | 0.6% | | | |
| H5 | Omnipaque/ | 63127.00 | 0.00 | |
| | 0.0% | | | |

TABLE 3
Neuraminidase Inhibition of HEXABRIX ®, Visipaque and Omnipaque
on A/Maryland/04/2011

| | Sample Name/ | Average | Percent Drug | EC50/IC50 |
|---|---|---|---|---|
| Standard | Concentration | Fluorescence | (%) | (% Drug) |
| A1 | HEXABRIX ®/ | 12150.00 | 37.50 | 12.25 |
| | 37.5% | | | |
| B1 | HEXABRIX ®/ | 26310.00 | 18.80 | |
| | 18.8% | | | |
| C1 | HEXABRIX ®/ | 40316.00 | 9.40 | |
| | 9.4% | | | |
| D1 | HEXABRIX ®/ | 55830.00 | 4.70 | |
| | 4.7% | | | |
| E1 | HEXABRIX ®/ | 59789.00 | 2.30 | |
| | 2.3% | | | |
| F1 | HEXABRIX ®/ | 66168.00 | 1.20 | |
| | 1.2% | | | |
| G1 | HEXABRIX ®/ | 69256.00 | 0.60 | |
| | 0.6% | | | |
| H1 | HEXABRIX ®/ | 77539.00 | 0.00 | |
| | 0.0% | | | |
| A3 | Visipaque/ | 17054.00 | 37.50 | 27.36 |
| | 37.5% | | | |
| B3 | Visipaque/ | 31535.00 | 18.80 | |
| | 18.8% | | | |
| C3 | Visipaque/ | 42311.00 | 9.40 | |
| | 9.4% | | | |
| D3 | Visipaque/ | 57679.00 | 4.70 | |
| | 4.7% | | | |
| E3 | Visipaque/ | 64086.00 | 2.30 | |
| | 2.3% | | | |
| F3 | Visipaque/ | 66253.00 | 1.20 | |
| | 1.2% | | | |
| G3 | Visipaque/ | 72107.00 | 0.60 | |
| | 0.6% | | | |
| H3 | Visipaque/ | 77867.00 | 0.00 | |
| | 0.0% | | | |
| A5 | Omnipaque/ | 15635.00 | 37.50 | 12.02 |
| | 37.5% | | | |
| B5 | Omnipaque/ | 27464.00 | 18.80 | |
| | 18.8% | | | |
| C5 | Omnipaque/ | 43772.00 | 9.40 | |
| | 9.4% | | | |
| D5 | Omnipaque/ | 58808.00 | 4.70 | |
| | 4.7% | | | |
| E5 | Omnipaque/ | 68133.00 | 2.30 | |
| | 2.3% | | | |
| F5 | Omnipaque/ | 73088.00 | 1.20 | |
| | 1.2% | | | |
| G5 | Omnipaque/ | 77645.00 | 0.60 | |
| | 0.6% | | | |
| H5 | Omnipaque/ | 81141.00 | 0.00 | |
| | 0.0% | | | |

TABLE 4
Oseltamivir on A/California/07/2009

| | | | | EC50/ |
|---|---|---|---|---|
| | | | Percent | IC50 |
| | | Average | Drug | (nM |
| Standard | Sample Name | Fluorescence | (%) | Drug) |
| A3 | Oseltamivir 50,000 nM | 365.00 | 50000.00 | 0.41 |
| B3 | Oseltamivir 5,000 nM | 179.00 | 5000.00 | |
| C3 | Oseltamivir 500 nM | 354.00 | 500.00 | |
| D3 | Oseltamivir 50 nM | 1215.00 | 50.00 | |
| E3 | Oseltamivir 5 nM | 7802.00 | 5.00 | |
| F3 | Oseltamivir 0.5 nM | 32060.00 | 0.50 | |
| G3 | Oseltamivir 0.05 nM | 56340.00 | 0.05 | |
| H3 | Oseltamivir 0.005 nM | 66385.00 | 0.01 | |

TABLE 5
Oseltamivir on A/Maryland/04/2011

| | | | | EC50/ |
|---|---|---|---|---|
| | | | Percent | IC50 |
| | | Average | Drug | (nM |
| Standard | Sample Name | Fluorescence | (%) | Drug) |
| A1 | Oseltamivir 50,000 nM | 1100.00 | 50000.00 | 196.07 |
| B1 | Oseltamivir 5,000 nM | 3256.00 | 5000.00 | |
| C1 | Oseltamivir 500 nM | 23318.00 | 500.00 | |
| D1 | Oseltamivir 50 nM | 64948.00 | 50.00 | |
| E1 | Oseltamivir 5 nM | 83140.00 | 5.00 | |
| F1 | Oseltamivir 0.5 nM | 80170.00 | 0.50 | |
| G1 | Oseltamivir 0.05 nM | 80182.00 | 0.05 | |
| H1 | Oseltamivir 0.005 nM | 81358.00 | 0.01 | |

The results of the NIA assay are summarized in Table 6 below.

TABLE 6
IC₅₀ values for three Samples for an H1N1 Oseltamivir resistant and
sensitive influenza viruses

| | | A/Maryland/04/2011 |
|---|---|---|
| | A/California/07/2009 | H275Y |
| | H1N1 | H1N1 |
| Sample | Oseltamivir Sensitive | Oseltamivir Resistant |
| HEXABRIX ® | 17.04% | 12.25% |
| Visipaque | 20.59% | 27.36% |
| Omnipaque | 13.17% | 12.02% |
| Oseltamivir | 0.41 nM | 196.07 nM |

### Example 6

HEXABRIX®, Vispaque and Omnipaque were tested in a plaque inhibition assay (PIA) using MDCK cells in the presence of TPCK trypsin.

The CM samples were stored in clear glass or plastic bottles at room temperature until use. The three stock CM samples tested are shown in Table 7 below.

TABLE 7
Stock CM Samples

| | | Starting |
|---|---|---|
| Sample | Details | Concentration |
| HEXABRIX ® | Tyco Healthcare, Mallinckrodt, | 100% |
| (Ioxaglic acid) | Cat. 5505-21, Lot A0231, | |
| | Exp January 2014, 200 ml | |
| Visipaque | GE Healthcare, Cat. NDC | 100% |
| (iodixanol) | 0407222316, Lot 12288359, | |
| | Exp 08 Nov. 2016, 50 ml | |
| Omnipaque | GE Healthcare, Cat. 0407141310, Lot | 100% |
| (iohexol) | 12212550, Exp 27 Sep. 2016, 10 ml | |

Dilutions of each CM sample were made in tissue culture media (Dulbecco's Modified Eagle media with 0.1 μg TPCK trypsin and antibiotic) at concentrations of 30, 15, 7.5, 3.75, 1.88 and 0.9% of the stock contrast medias shown in Table 7.

Three strains of influenza virus were tested in the presence or absence of each CM media and in the presence or absence of Oseltamivir. The three influenza strains are identified below:
Influenza Virus A/California/07/2009 H1N1, Sensitive to Oseltamivir, CDC ID 2009712112, VIRAPUR Lot G1121C1 (Host Cell Line: Madin-Darby canine kidney (MDCK)).
Influenza Virus A/Maryland/04/2011 H1N1, Resistant to Oseltamivir, H275Y, CDC ID 2011767959, VIRAPUR Lot E1218B (Host Cell Line: MDCK).
Influenza Virus A/Vietnam/1203/2004 reassortant, H5N1, VIRAPUR lot A1206B (Host Cell Line: MDCK).

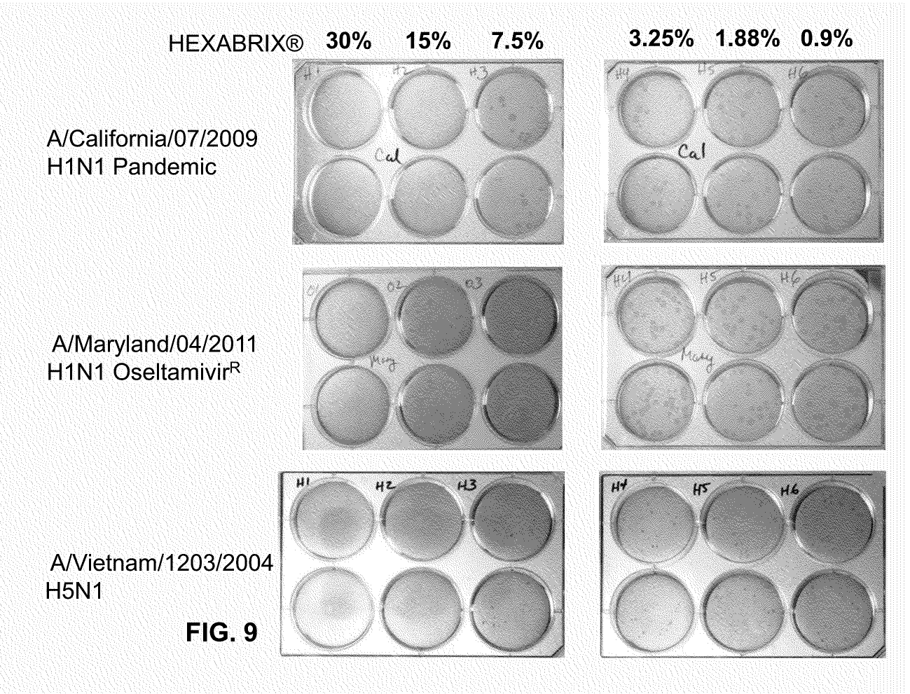

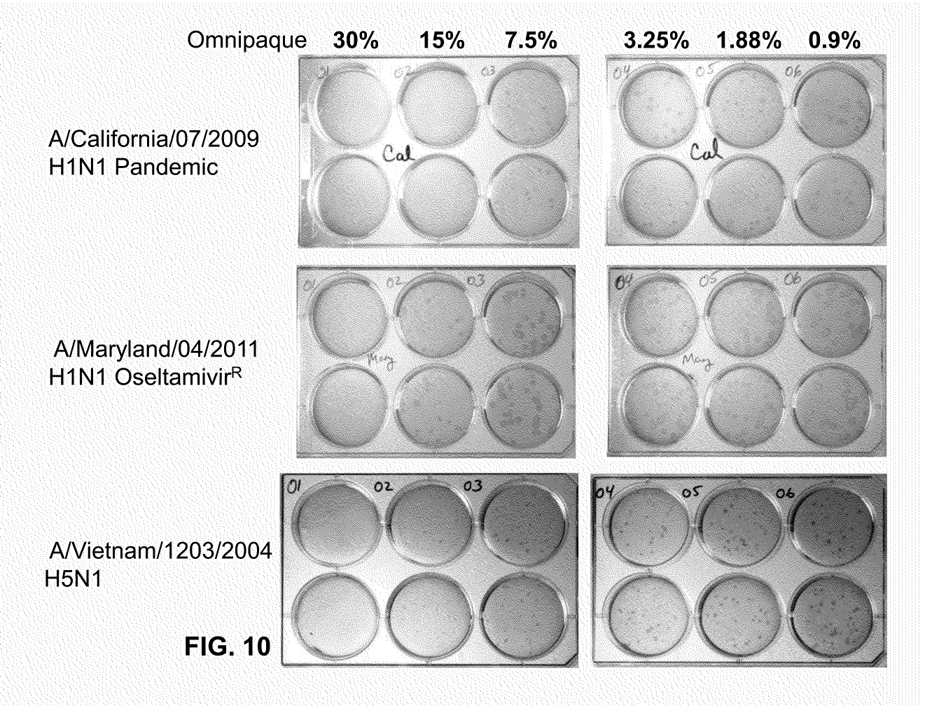

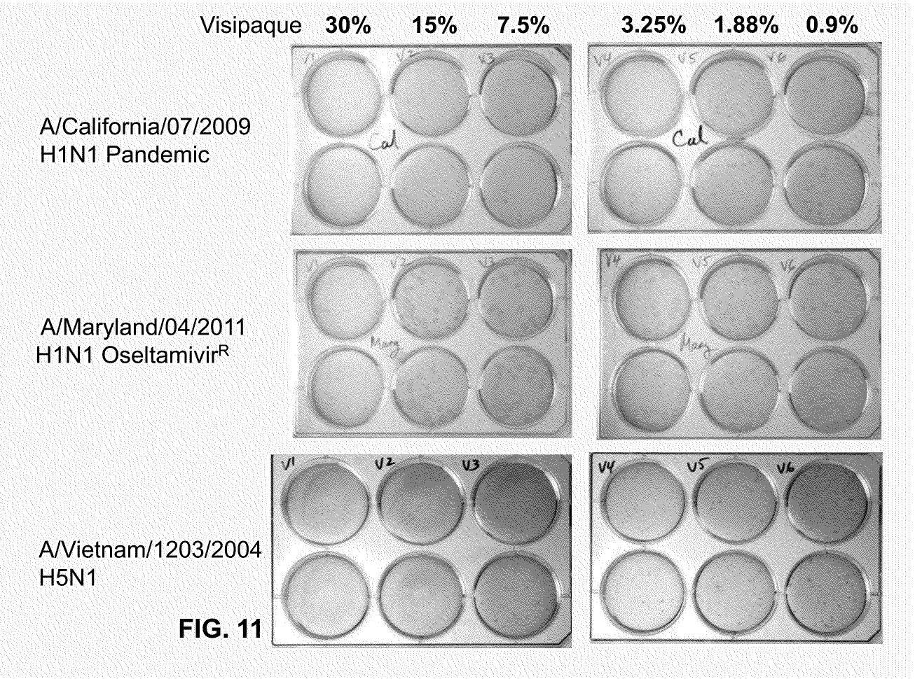

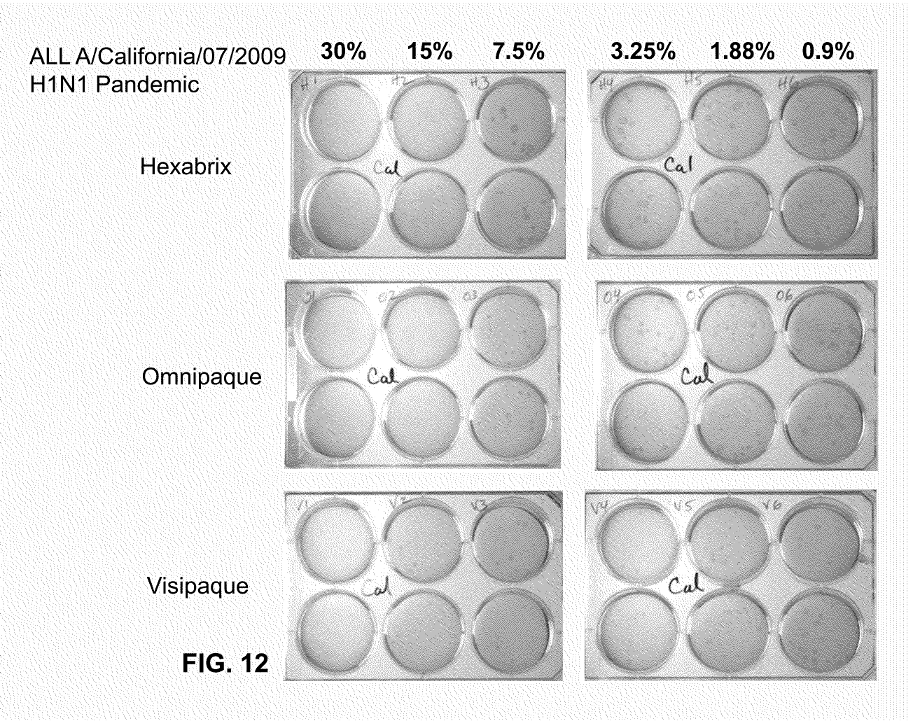

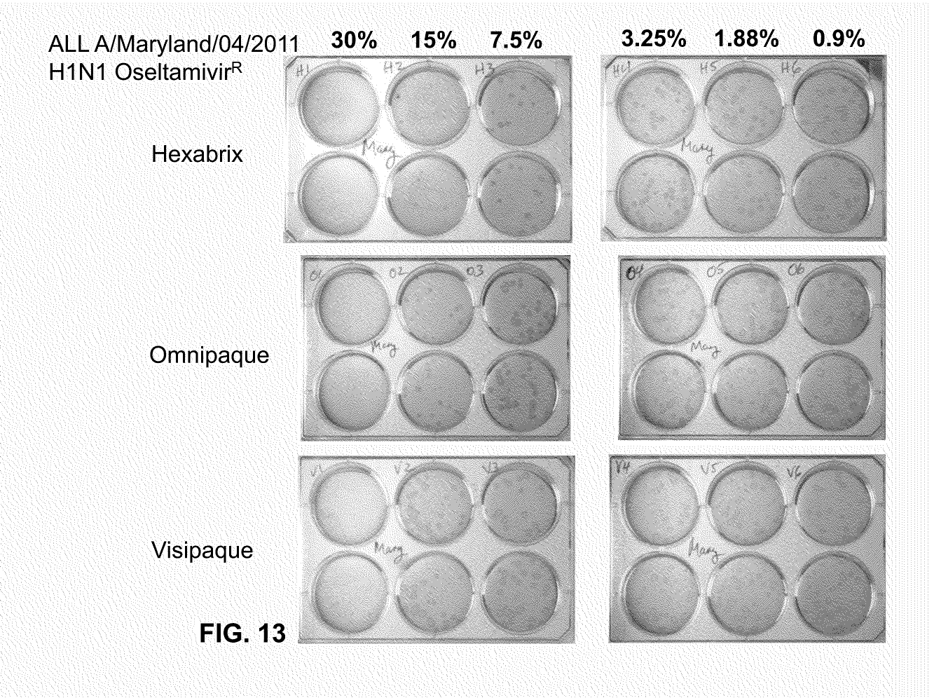

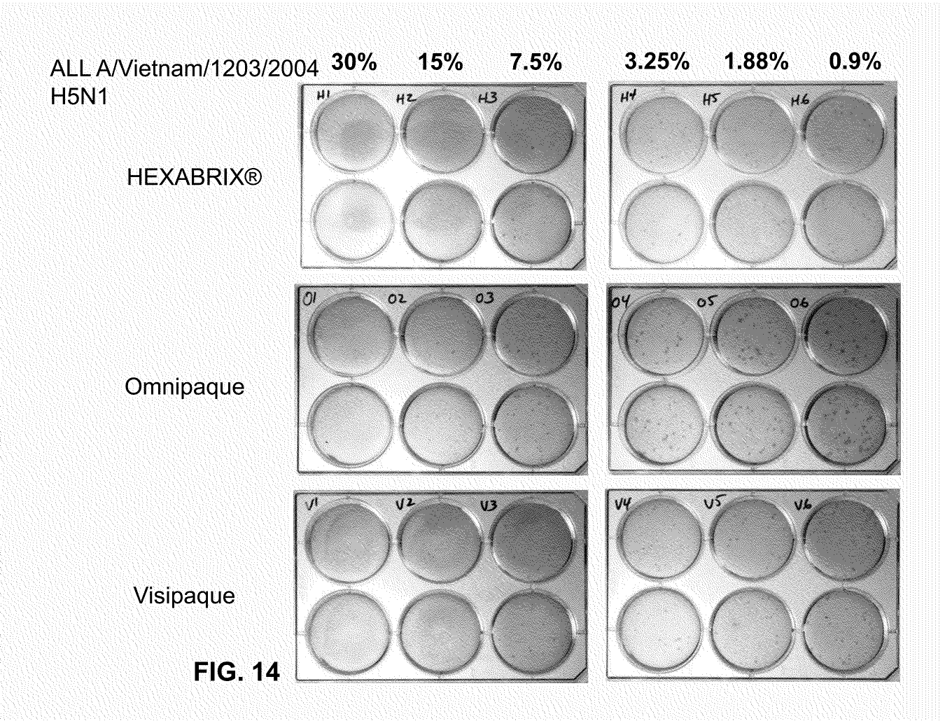

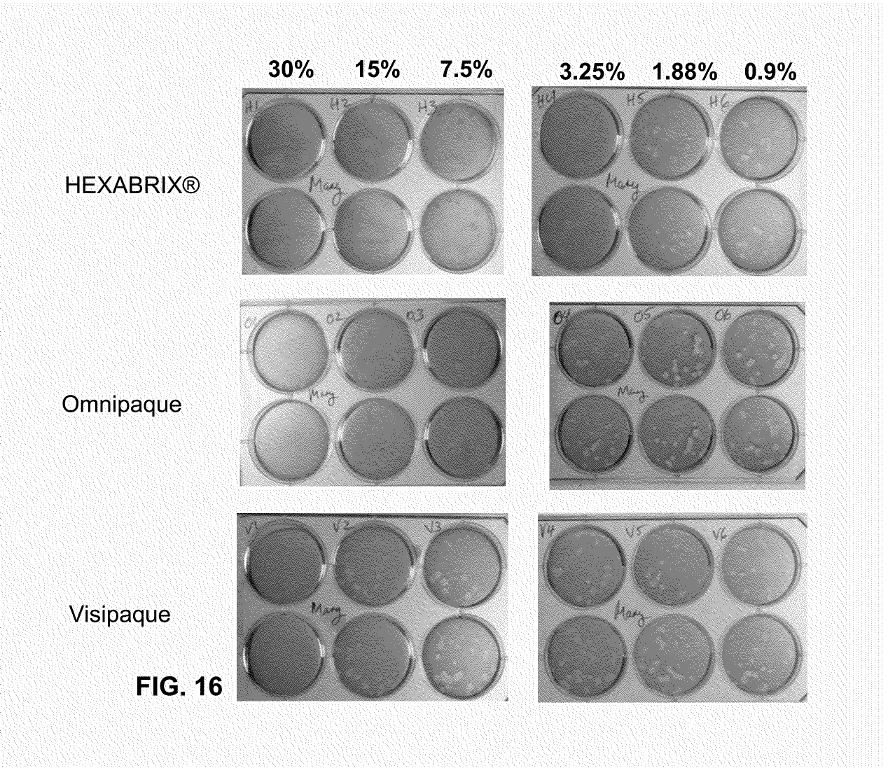

Six-well plates were seeded on Day 1 with a known concentration of MDCK cells and incubated overnight at 37° C. On Day 2, CM sample dilutions were added to duplicate wells of the 6-well plates and further incubated for one hour at which point approximately 50 plaque forming units of virus in a volume of 80 μl were added to each well. Virus was allowed to absorb to the cells for 2 hours before the solution and any remaining virus was removed. An agarose solution containing dilutions of each CM sample or the appropriate controls was added to the infected cells in the appropriate wells. Plates were returned to incubate at 37° C. for three days. On Day 5, cultures were stained for 2 hours with Methylthiazolyldiphenyl-tetrazolium bromide (MTT) and photographs were taken of each plate (FIGS. 9-15). Under these conditions, MTT stains the plaques blue and the monolayers remain unstained. Plaques were counted and numbers recorded. After the plaques were counted, A/Maryland infected plates were returned to the incubator for additional 16 hour incubation for viable cell staining (FIG. 16).

Cells were expected to remain viable during the 48 hour infection period in order for influenza virus plaques to develop. Cell monolayers were briefly stained at 72 hours with MTT to primarily visualize plaques, but as the incubation is extended another 16 hours, MTT will stain viable cells blue and non-viable cells will remain unstained. MTT is converted to a blue color by the mitochondrial dehydrogenase of living cells.

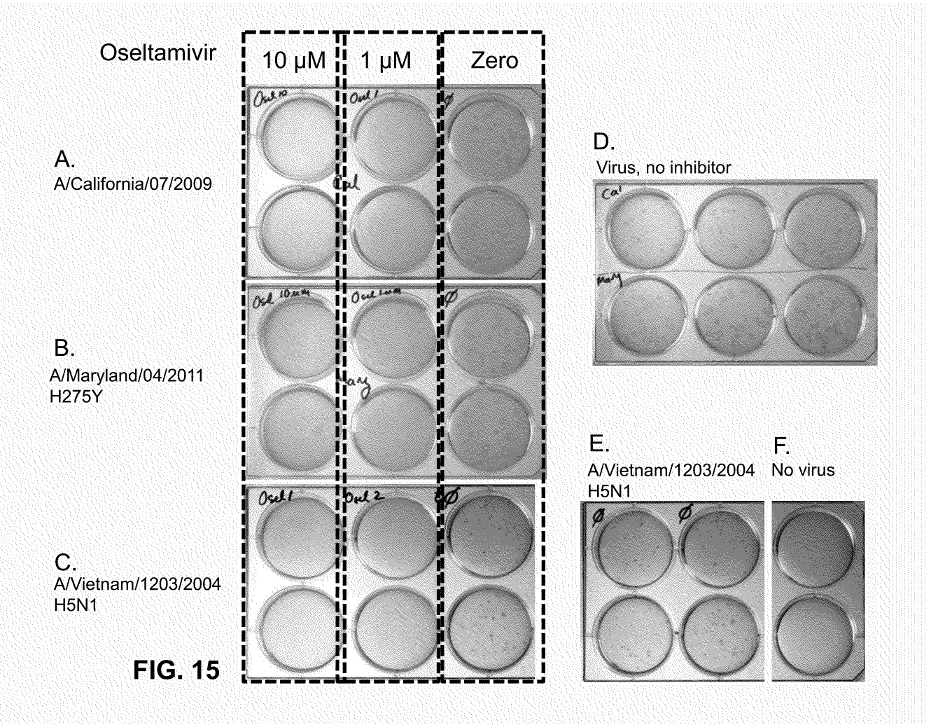

Oseltamivir was included in each virus test (e.g., see FIG. 15). Influenza A/California 07/2009 and A/Vietnam/1203/2004 H5N1 are sensitive to this drug to 1 μM, but A/Maryland/04/2011 has a mutation in the neuraminidase gene (H275Y) making it only partially sensitive to oseltamivir in this assay.

### Results

Images of the results of the PIA assay are shown in FIGS. 9-16 and the results for the 2 hour MTT staining are summarized in Tables 8-10. Tables 8-10 show the number of viral plaques counted in each duplicate assay for the indicated treatments.

TABLE 8
Number of A/California/07/2009 Viral Plaques after Drug Treatment

| CM | Compound | Compound | Compound | | Oseltamivir |
|---|---|---|---|---|---|
| Dilutions | HEXABRIX ® | Omnipaque | Visipaque | No treatment | Positive control |
| 30% | 0, 0 | 0, 0 | 0, 0 | 36, 26 | 10 μm |
| | | | | 20, 21, 28 | 0, 0 |
| 15% | 0, 0 | 0, 0 | 7, 8 | | 1 μM |
| | | | | | 0, 0 |
| 7.5% | 13, 15 | 14, 17 | 17, 16 | | |
| 3.75% | 16, 19 | 21, 16 | 13, 17 | | |
| 1.87% | 21, 25 | 23, 22 | 19, 26 | | |
| 0.9% | 23, 21 | 14, 25 | 16, 24 | | |

TABLE 9
Number of A/Maryland/04/2011 Viral Plaques after Drug Treatment

| Sample | Compound | Compound | Compound | | Oseltamivir |
|---|---|---|---|---|---|
| Dilutions | HEXABRIX ® | Omnipaque | Visipaque | No treatment | Positive control |
| 30% | 0, 0 | 0, 0 | 10, 14 | 39, 36, 36 | 10 μm |
| | | | Small | | 7, 4 |
| | | | plaques | | |
| 15% | 11, 9 | 16, 20 | 26, 27 | | 1 μM |
| | | | | | 33, 24 |
| 7.5% | 18, 18 | 27, 31 | 18, 35 | | |
| 3.75% | 29, 35 | 32, 39 | 24, 33 | | |
| 1.87% | 33, 23 | 31, 36 | 29, 34 | | |
| 0.9% | 34, 30 | 31, 29 | 22, 33 | | |

TABLE 10
Number of A/Vietnam/1203/2004 reassortant Viral Plaques after Drug Treatment

| Sample | Compound | Compound | Compound | | Oseltamivir |
|---|---|---|---|---|---|
| Dilutions | HEXABRIX ® | Omnipaque | Visipaque | No treatment | Positive control |
| 30% | 0, 0 | 0, 0 | 0, 0 | 35, 27, 40, | 10 μm |
| | | | | 48 | 0, 0 |
| 15% | 4, 7 | 24, 22 | 11, 13 | | 1 μM |
| | | | | | 0, 0 |
| 7.5% | 20, 26 | 41, 41 | 23, 29 | | |
| 3.75% | 31, 37 | 40, 50 | 40, 32 | | |
| 1.87% | 34, 37 | 48, 41 | 26, 34 | | |
| 0.9% | 40, 46 | 46, 52 | 57, 44 | | |

FIG. 16 shows photos of the assay plates stained with MTT for 18 hours. At the highest Cm sample dilution, MTT staining is reduced in HEXABRIX® and Omnipaque treatment relative to Visipaque treated cells. This may indicate that cell viability is affected by the high (e.g., 30%) drug concentration of HEXABRIX® and Omnipaque. Visipaque did not appear to have the same effect on MTT staining at high concentrations.

It is inconclusive on whether the function of trypsin is affected by the Samples in this assay. Trypsin inactivity would inhibit plaque size and formation by limiting virus spread.

Oseltamivir showed inhibitory effects on pandemic A/California/07/2009 H1N1 and the reassortant of A/Vietnam/1203/2004 H5N1, but the drug resistant mutant A/Maryland/04/2011 displayed reduced oseltamivir susceptibility in this assay. These results were expected with this control drug. HEXABRIX® showed inhibitory effects on all 3 viruses, inhibiting plaques with a concentration as low as 7.5%. Omnipaque showed plaque inhibitory effects at 30% and 15%, but MTT staining at these two dilutions was also impacted and the overall blue stain at these dilutions was diminished. Visipaque shoed inhibitory effects on A/California and A/Vietnam, inhibiting plaques at concentrations as low as 15%. Visipaque also showed little effect on MTT staining at higher concentrations. Plaque inhibition by all three CM samples was most pronounced against H5N1 in this assay. In some plates infected with A/Maryland, very small plaques (e.g., much smaller than controls) were visible even at 30% Visipaque.

### Materials.

TABLE 11
Materials

| | |
|---|---|
| 1. | HyClone DMEM/High Glucose media, Catalog Nr. SH30022.02, |
| | Lot AYF160966, Exp |
| 2 | Seradign Fetal Bovine Serum, Catalog Nr. 1400-500, Lot 168A10R, |
| | Exp June 2015 |
| 3. | Mediatech Trypsin EDTA, 1X, Catalog Nr. 25052-CV, Lot 25052402 |
| 4. | Mediatech Antibiotic Antimycotic Solution, Catalog Nr. 30-004-CI, |
| | Lot 30004110 |
| 5. | TPCK Trypsin Sigma, T8802, Lot 107K7014 |

### Example 8

### Embodiments

A1. A method of preventing or slowing infection with an influenza virus or of treating influenza in a mammal, comprising providing or administering to a mammal in need thereof a composition comprising one or more X-ray contrast media compounds in an amount sufficient to prevent or slow said influenza infection or in an amount sufficient to treat said influenza.

A2. A method of preventing or slowing infection with an influenza virus or of treating influenza in a mammal, comprising providing or administering to a mammal in need thereof a composition comprising a compound of Formula (I) or Formula (II) or a pharmaceutically acceptable salt or ester thereof in an amount sufficient to prevent or slow said influenza infection or in an amount sufficient to treat said influenza, wherein Formula (I) has the following structure:

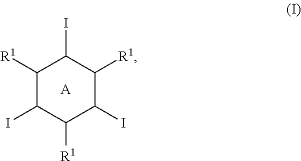

Formula (II) has the following structure:

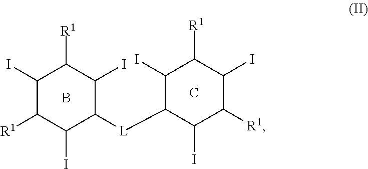

each R¹ is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl;

L is null or a linker comprising one or more R²;

each R² is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl; and

each ring of A, B and C of Formula (I) and (II) is independently aromatic, partially unsaturated or fully saturated.

A3. The method of embodiments A1 or A2, wherein the composition is administered to a mucous membrane.

A4. The method of any one of embodiments A1 to A3, wherein the composition is administered or provided intranasally.

A5. The method of any one of embodiments A1 to A3, wherein the composition is administered or provided to one or more of the lungs, bronchi, and trachea.

A5.1. The method of any one of embodiments A1 to A5, wherein the composition is administered as an inhalant.

A6. The method of any one of embodiments A1 to A3, wherein the composition is administered orally or buccally.

A6.1. The method of embodiment A6, wherein the composition is administered as a lozenge.

A7. The method of any one of embodiments A1 to A3, wherein the composition is administered parenterally.

A8. The method of any one of embodiments A1 to A3, wherein the composition is administered topically.

A9. The method of any one of embodiments A1 to A8, wherein said influenza virus is selected from influenza type A, influenza type B, and influenza type C.

A10. The method of any embodiment A8, wherein said influenza virus is Influenza type A.

A11. The method of any one of embodiments A1 to A10, wherein said influenza virus is selected from a swine origin influenza virus, H1N1 and H3N2 serotypes.

A12. The method of any one of embodiments A1 to A11, wherein the composition comprises the one or more compounds in a concentration of 150 mg I/mL to 350 mg I/mL.

A13. The method of any one of embodiments A1 to A11, wherein the composition comprises the one or more compounds in a concentration in excess of 350 mg I/mL.

A14. The method of any one of embodiments A1 to A11, wherein the composition comprises the one or more compounds in a concentration of up to 150 mg I/mL.

A15. The method of any one of embodiments A1 to A14, wherein the X-ray contrast media compound is selected from the group consisting of either a monomeric or dimeric, nonionic or ionic contrast media.

A16. The method of any one of embodiments A1 to A15, wherein the X-ray contrast media comprises triiodinated, completely or partially substituted, benzene moieties.

A17. The method of any one of embodiments A1 to A16, wherein the X-ray contrast media compound is selected from the group consisting of iopamidol, ioversol, iopromide, iohexol, iothalamate (iothalamic acid), diatrizoate, ioxaglate (ioxaglic acid), iodipamide, iodixanol, iopanoic acid, sodium tyropanoate (BILOPAQUE®), iotrolan, acetrizoate sodium, bunamidiodyl sodium, diatrizoate sodium, iobenzamic acid, iocarmic acid, iocetamic acid, iodamide, iodophthalein sodium, ioglycamic acid, iomeglamic acid, iopental, iophenoxic acid, iopromide, ipronic acid, ioxilan, ipodate, meglumine acetrizoate, meglumine diatrizoate, metrizamide, metrizoic acid, phenobutiodil, phentetiothalein sodium, tyropanoate sodium, and combinations thereof.

A18. The method of any one of embodiments A1 to A17, wherein the X-ray contrast media compound is selected from the group consisting of iopamidol, ioversol, iopromide, iohexol, iothalamate (iothalamic acid), diatrizoate, ioxaglate or combinations thereof.

A19. Use of any compound as described in any one of embodiments A1 to A18, for the preparation of a medicament.

A20. The use of the medicament of embodiment 21, wherein the medicament is for the prevention, treatment or reduction of severity of influenza infection or influenza symptoms.

B1. A method of preventing or slowing an influenza virus infection or of treating the influenza virus infection in a mammal, comprising providing or administering to a mammal in need thereof a composition comprising an X-ray contrast media compound in an amount sufficient to prevent or slow said influenza infection or in an amount sufficient to treat said influenza.

B2. A method of treating a mammal having an influenza virus infection, suspected of having an influenza virus infection or at risk of havening an influenza virus infection comprises providing or administering to the mammal a composition comprising or consisting of an X-ray contrast media compound in an amount sufficient to prevent or slow said influenza virus infection or in an amount sufficient to treat said influenza.

B3. The method of embodiment B1 or B2, wherein the X-ray contrast media compound has a formula selected from the group consisting of Formula (I) or Formula (II) or a pharmaceutically acceptable salt or ester thereof, wherein Formula (I) has the following structure:

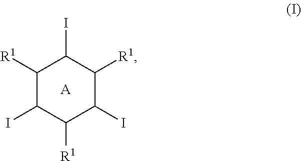

Formula (II) has the following structure:

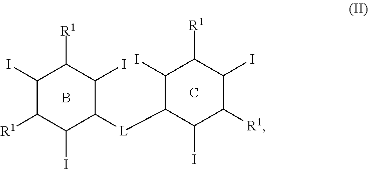

each R¹ is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl;

L is null or a linker comprising one or more R²;

each R² is independently selected from the group consisting of hydrogen, halogen, nitro, amino, ketone, ester, amide, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl; and

each ring of A, B and C of Formula (I) and (II) is independently aromatic, partially unsaturated or fully saturated.

B3.1. The method of embodiment B3, wherein each ring of A, B and C of Formula (I) and (II) is aromatic.

B3.2. The method of embodiment B3 or B3.1, wherein R² is independently selected from the group consisting of amino, ketone, ester, amide, optionally substituted C1-C5 alkyl, optionally substituted C2-C5 alkenyl, optionally substituted C2-C5 alkynyl, acyl, acyloxy, alkoxy, aryl, arylalkoxy carbonyl, alkoxy carbonylacyl, aminocarbonyl, aminocarboyloxy, and carboxy.

B4. The method of any one of embodiments B1 or B3.2, wherein the composition is administered to a mucous membrane.

B5. The method of any one of embodiments B1 to B3.2, wherein the composition is administered intranasally.

B6. The method of any one of embodiments B1 to B3.2, wherein the composition is administered to one or more of the lungs, bronchi, and trachea.

B7. The method of any one of embodiments B1 to B3.2, wherein the composition is administered as an inhalant.

B8. The method of any one of embodiments B1 to B3.2, wherein the composition is administered orally or buccally.

B9. The method of any one of embodiments B1 to B3.2, wherein the composition is administered as a lozenge.

B10. The method of any one of embodiments B1 to B3.2, wherein the composition is administered parenterally.

B11. The method of any one of embodiments B1 to B3, wherein the composition is administered topically.

B12. The method of any one of embodiments B1 to B11, wherein the influenza virus infection is an infection by an influenza type A, influenza type B, or influenza type C virus.

B13. The method of any embodiment B12, wherein the influenza virus infection is an infection by an Influenza type A virus.

B14. The method of embodiment B13, wherein the Influenza type A virus is selected from a swine origin influenza virus, H1N1 and H3N2 serotype.

B14.1. The method of embodiment B14, wherein the Influenza type A virus is H1N1.

B15. The method of any one of embodiments B1 to B14.1, wherein the composition comprises the X-ray contrast media compound in a concentration of 150 mg I/mL to 350 mg I/mL.

B16. The method of any one of embodiments B1 to B14.1, wherein the composition comprises the X-ray contrast media compound in a concentration in excess of 350 mg I/mL.

B17. The method of any one of embodiments B1 to B14.1, wherein the composition comprises the X-ray contrast media compound in a concentration of up to 150 mg I/mL.

B18. The method of any one of embodiments B1 to B14.1, wherein the X-ray contrast media compound is selected from the group consisting of either a monomeric or dimeric, nonionic or ionic contrast media.

B19. The method of any one of embodiments B1 to B18, wherein the X-ray contrast media compound is selected from the group consisting of iopamidol, ioversol, iopromide, iohexol, iothalamate (iothalamic acid), diatrizoate, ioxaglate), iodipamide, iodixanol, iopanoic acid, sodium tyropanoate, iotrolan, acetrizoate sodium, bunamidiodyl sodium, diatrizoate sodium, iobenzamic acid, iocarmic acid, iocetamic acid, iodamide, iodophthalein sodium, ioglycamic acid, iomeglamic acid, iopental, iophenoxic acid, iopromide, ipronic acid, ioxilan, ipodate, meglumine acetrizoate, meglumine diatrizoate, metrizamide, metrizoic acid, phenobutiodil, phentetiothalein sodium, tyropanoate sodium, and combinations thereof.

B20. The method of any one of embodiments B1 to B18, wherein the X-ray contrast media compound is selected from the group consisting of iopamidol, ioversol, iopromide, iohexol, iothalamate, diatrizoate, ioxaglate and combinations thereof.

C1. A composition comprising an X-ray contrast media compound for use as a medicament for treating or preventing an influenza virus infection in a mammal, wherein the mammal has, is suspected of having, or is at risk of an influenza virus infection.

C2. A composition comprising an X-ray contrast media compound for the treatment of or prevention of an influenza virus infection in a mammal, wherein the mammal has, is suspected of having, or is at risk of an influenza virus infection.

C3. The composition of embodiment C1 or C2, wherein the X-ray contrast media compound has a formula selected from the group consisting of Formula (I) or Formula (II) or a pharmaceutically acceptable salt or ester thereof, wherein Formula (I) has the following structure:

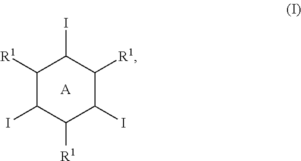

Formula (II) has the following structure:

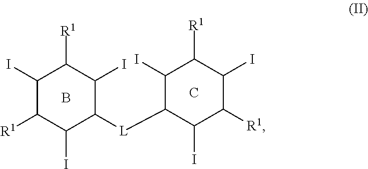

each R¹ is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl; L is null or a linker comprising one or more R²;

each R² is independently selected from the group consisting of hydrogen, halogen, nitro, amino, ketone, ester, amide, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl; and

each ring of A, B and C of Formula (I) and (II) is independently aromatic, partially unsaturated or fully saturated.

C4. The composition of any one of embodiments C1 to C3, wherein the amount of the X-ray contrast media compound is sufficient to prevent, slow or terminate an influenza infection.

C5. The composition of any one of embodiments C1 to C4, wherein the composition is administered to the mammal.

C6. The composition of any one of embodiments C1 or C5, wherein the composition is administered to a mucous membrane.

C7. The composition of any one of embodiments C1 to C5, wherein the composition is administered intranasally.

C8. The composition of any one of embodiments C1 to C5, wherein the composition is administered to one or more of the lungs, bronchi, and trachea.

C9. The composition of any one of embodiments C1 to C5, wherein the composition is administered as an inhalant.

C10. The composition of any one of embodiments C1 to C5, wherein the composition is administered orally or buccally.

C11. The composition of any one of embodiments C1 to C5, wherein the composition is administered as a lozenge.

C12. The composition of any one of embodiments C1 to C5, wherein the composition is administered parenterally.

C13. The composition of any one of embodiments C1 to C5, wherein the composition is administered topically.

C14. The composition of any one of embodiments C1 to C13, wherein the influenza virus infection is an infection by an influenza type A, influenza type B, or influenza type C virus.

C15. The composition of any embodiment C14, wherein the influenza virus infection is an infection by an Influenza type A virus.

C16. The composition of embodiment C15, wherein the Influenza type A virus is selected from a swine origin influenza virus, H1N1 and H3N2 serotype.

C17. The composition of embodiment C16, wherein the Influenza type A virus is H1N1.

C18. The composition of any one of embodiments C1 to C17, wherein the composition comprises the X-ray contrast media compound in a concentration of 150 mg I/mL to 350 mg I/mL.

C19. The composition of any one of embodiments C1 to C7, wherein the composition comprises the X-ray contrast media compound in a concentration in excess of 350 mg I/mL.

C20. The composition of any one of embodiments C1 to C17, wherein the composition comprises the X-ray contrast media compound in a concentration of up to 150 mg I/mL.

C21. The composition of any one of embodiments C1 to C20, wherein the X-ray contrast media compound is selected from the group consisting of either a monomeric or dimeric, nonionic or ionic contrast media.

C22. The composition of any one of embodiments C1 to C21, wherein the X-ray contrast media compound is dimeric.

C23. The composition of any one of embodiments C1 to C21, wherein the X-ray contrast media compound is monomeric.

C24. The composition of any one of embodiments C1 to C23, wherein the X-ray contrast media compound is ionic.

C25. The composition of any one of embodiments C1 to C23, wherein the X-ray contrast media compound is non-ionic.

C26. The composition of any one of embodiments C1 to C21, wherein the X-ray contrast media compound is selected from the group consisting of iopamidol, ioversol, iopromide, iohexol, iothalamate (iothalamic acid), diatrizoate, ioxaglate), iodipamide, iodixanol, iopanoic acid, sodium tyropanoate, iotrolan, acetrizoate sodium, bunamidiodyl sodium, diatrizoate sodium, iobenzamic acid, iocarmic acid, iocetamic acid, iodamide, iodophthalein sodium, ioglycamic acid, iomeglamic acid, iopental, iophenoxic acid, iopromide, ipronic acid, ioxilan, ipodate, meglumine acetrizoate, meglumine diatrizoate, metrizamide, metrizoic acid, phenobutiodil, phentetiothalein sodium, tyropanoate sodium, and combinations thereof.

C27. The composition of any one of embodiments C1 to C6, wherein the X-ray contrast media compound is selected from the group consisting of iopamidol, ioversol, iopromide, iohexol, iothalamate, diatrizoate, ioxaglate and combinations thereof.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It will be understood by those of skill in the art that numerous and various modifications can be made without departing from the spirit of the technology. Therefore, it should be clearly understood that the forms of the technology are illustrative only and are not intended to limit the scope of the technology.

What is claimed is:

1. A method of preventing or slowing infection with an influenza virus or of treating influenza in a mammal, comprising providing or administering to a mammal in need thereof a composition comprising one or more X-ray contrast media compounds in an amount sufficient to prevent or slow said influenza infection or in an amount sufficient to treat said influenza.

2. The method of claim 1, wherein the one or more X-ray contrast media compounds have a formula selected from the group consisting of Formula (I), Formula (II) and a pharmaceutically acceptable salt or ester thereof, wherein Formula (I) has the following structure: 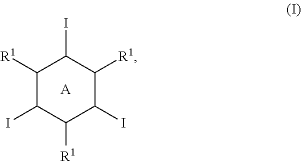
Formula (II) has the following structure: 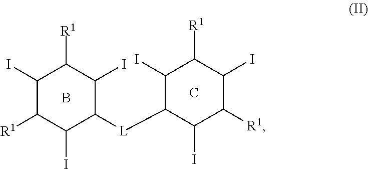
each R¹ is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl;
L is null or a linker comprising one or more R²;
each R² is independently selected from the group consisting of hydrogen, halogen, nitro, amino, hydroxyl, cyano, optionally substituted C₁-C₂₄ alkyl, optionally substituted C₂-C₂₄ alkenyl, optionally substituted C₂-C₂₄ alkynyl, acyl, acyloxy, alkyloxycarbonyloxy, aryloxycarbonyloxy, cycloalkyl (including for example, cyclohexylcarbinol), cycloalkenyl, alkoxy, cycloalkoxy, aryl, heteroaryl, arylalkoxy carbonyl, alkoxy carbonylacyl, aminocarbonyl, aminocarboyloxy, azido, phenyl, cycloalkylacyl, alkylthio, arylthio, oxysulfonyl, carboxy, thio, sulfoxide, sulfone, sulfonate esters, thiocyano, boronic acids and esters, and halogenated alkyl including polyhalogenated alkyl; and
each ring of A, B and C of Formula (I) and (II) is independently aromatic, partially unsaturated or fully saturated.

3. The method of claim 1, wherein the composition is administered or provided to a mucous membrane.

4. The method of claim 3, wherein the composition is administered or provided intranasally.

5. The method of claim 3, wherein the composition is administered or provided to one or more of the lungs, bronchi, and trachea.

6. The method of claim 1, wherein the composition is administered orally or buccally.

7. The method of claim 1, wherein the composition is administered parenterally.

8. The method of claim 1, wherein the composition is administered topically.

9. The method of claim 1, wherein said influenza virus is selected from influenza type A, influenza type B, and influenza type C.

10. The method of claim 9, wherein said influenza virus is Influenza type A.

11. The method of claim 1, wherein said influenza virus is selected from a swine origin influenza virus, H1N1 and H3N2 serotypes.

12. The method of claim 1, wherein the composition comprises the one or more X-ray contrast media compounds in a concentration of about 150 mg I/mL to about 350 mg I/mL.

13. The method of claim 1, wherein the composition comprises the one or more X-ray contrast media compounds in a concentration of at least 350 mg I/mL.

14. The method of claim 1, wherein the composition comprises the one or more X-ray contrast media compounds in a concentration of up to 150 mg I/mL.

15. The method of claim 1, wherein the composition is administered as an inhalant.

16. The method of claim 1, wherein the composition is administered as a lozenge.

17. The method of claim 1, wherein the one or more X-ray contrast media compounds are a monomeric or dimeric contrast media.

18. The method of claim 1, wherein the one or more X-ray contrast media compounds are a nonionic or ionic contrast media.

19. The method of claim 1, wherein the one or more X-ray contrast media compounds comprise triiodinated, completely or partially substituted, benzene moieties.

20. The method of claim 1, wherein the one or more X-ray contrast media compounds are selected from the group consisting of iopamidol, ioversol, iopromide, iohexol, iothalamate, diatrizoate, ioxaglate, iodipamide, iodixanol, iopanoic acid, sodium tyropanoate, iotrolan, acetrizoate sodium, bunamidiodyl sodium, diatrizoate sodium, iobenzamic acid, iocarmic acid, iocetamic acid, iodamide, iodophthalein sodium, ioglycamic acid, iomeglamic acid, iopental, iophenoxic acid, iopromide, ipronic acid, ioxilan, ipodate, meglumine acetrizoate, meglumine diatrizoate, metrizamide, metrizoic acid, phenobutiodil, phentetiothalein sodium, tyropanoate sodium, and combinations thereof.

21. The method of claim 1, wherein the one or more X-ray contrast media compounds are selected from the group consisting of iopamidol, ioversol, iopromide, iohexol, iothalamate, diatrizoate, ioxaglate and combinations thereof.
